(19) United States
(12) Patent Application Publication
(10) Pub. No.: US 2015/0080851 A1
(43) Pub. Date: Mar. 19, 2015

(54) Universal Transvalvular Insertion Tool for Use with Hemostatic Cardiac Introducers and Method of Using the Same

(71) Applicant: Pressure Products Medical Supplies Inc., Santa Barbara, CA (US)

(72) Inventors: Paul Kurth, Santa Barbara, CA (US); Andrew Armour, Swarthmore, PA (US)

(73) Assignee: Pressure Products Medical Supplies Inc., Santa Barbara, CA (US)

(21) Appl. No.: 14/395,006

(22) PCT Filed: Apr. 24, 2013
(86) PCT No.: PCT/US13/38074

§ 371 Date: Oct. 16, 2014

Related U.S. Application Data
(60) Provisional application No. 61/638,527, filed on Apr. 26, 2012.

(51) Int. Cl. A61M 25/01; A61M 39/06

(52) U.S. Cl. CPC A61M 25/01; A61M 39/0693; A61M 2039/0626; A61M 2025/0188
USPC 604/507; 604/264; 604/164.02; 604/506

(57) ABSTRACT

The invention includes a universal transvalvular insertion tool for providing a passage through a multiplicity of differently designed hemostatically valved introducers, each having a valve within a valved body coupled to a cardiac introducer. The tool includes a longitudinally rigid sheath having a lumen and for insertion through the valve of any one of the multiplicity of differently designed hemostatically valved introducers to provide a substantially obstruction free path for delivery of a lead or vascular device through the valve and valved body to the introducer, the sheath having a length selected to extend through the valve without substantial deformation of the lumen of the sheath. A mechanism is provided for separating the rigid sheath to allow removal from the lead or device without removal of the transvalvular insertion tool over either end of the lead or vascular device.

### RELATED APPLICATIONS

The present application is related to U.S. Provisional Patent Application Ser. No. 61/638,321, filed on Apr. 25, 2012, which is incorporated herein by reference and to which priority is claimed pursuant to 35 USC 119.

### BACKGROUND

1. Field of the Technology

The disclosure relates to the field of accessories for use with hemostatic cardiac introducers.

2. Description of the Prior Art

Prior art Trans-Valvular Insertion (TVI) tools, are typically comprised of peel-away sheaths with a splitting means on the proximal end of the tool as shown in FIG. 1. The tool is configured to be inserted into a hemostatic valve. The hemostatic valve is comprised of a splittable valve body and a splittable or peel-away introducer sheath. The sheath peels apart on lines of weakness along its longitudinal length. The valve body is usually rigid and the sheath is a softer material attached to the valve body, both having some means of splitting in half. Inside the valve body is a membrane with a slit, which is normally closed and fluid tight. A tool or pacemaker lead is inserted through the slit in the membrane in the valve body and through the introducer connected to the distal end of the valve body to be delivered to a location in the heart.

Once the tool or lead is located in the heart, the introducer is pulled out of the vascular system and the introducer and hemostatic valve body are split apart and pulled off the lead. The lead's proximal connector, which is too large to fit through the introducer or valve, is connected to the pacemaker, which is then inserted into a subdermal chest pocket surgically created in the chest muscles.

The membrane seals on the lead and prevents air embolism. The embodiment shown in the upper portion of FIG. 1 is simply a peel-away sheath that has been partially split on its proximal end. The embodiment below is a peel away sheath with a pair of rigid plastic handles bonded to the proximal end of the sheath. The purpose of the TVI tool is to facilitate insertion of the pacemaker lead, which can be quite flexible or delicate, through the membrane valve and valve body. The TVI tool is inserted through the membrane in the valve body to open it to allow a lead to be placed through the membrane without damage to the lead. Some leads have delicate wiring coils or coatings on their exterior that are damaged if forced through the membrane in the process of being introduced through the introducer into the heart or when being pulled back through the membrane.

Since the introduction of the first splittable hemostatic introducer by Pressure Products Medical Supplies Inc., namely the SafeSheath, and the expiration of the patents thereon, there have arisen now many different designs for different kinds of splittable hemostatic introducers and valves on the market. The various designs are typically differentiated from each other in the details of their internal dimensions and relative placement of the valve with respect to other portions of the hemostatic valve assembly. As a result, the prior art TVI tools in FIG. 1 may be unusable in many hemostatic introducers, i.e. the TVI tool bottoms out in the valve body, distorts and otherwise pinches or frictionally engages the cardiac tool or lead disposed through the introducer. The distal end of the TVI tool contacts the transition between the introducer and bottom of the valve body and is crushed, bent, closed or otherwise impeded, because the TVI tool no longer “fits” the design or dimensions of each different kind of hemostatic valved introducer. As a result, a lead then placed through the TVI tool is blocked or damaged when forced through the distorted TVI tool.

Ye et. al. U.S. Pat. No. 7,993,305, incorporated herein by reference, is directed to a splittable valved introducer apparatus that is designed to introduce a medical device such as a lead or catheter into a patient's vasculature. FIGS. 1 and 2 in Ye explicitly depict the SafeSheath. Furthermore, the introducer assembly is designed to separate easily without disrupting the placement of the medical device during the removal of the introducer. Referring to FIG. 3 et. seq. in Ye, the valved introducer assembly 100 comprises a sheath 102 connected to a valve housing 104 with valve membrane 106. The longitudinal sheath 102 extends into the valve housing 104 while the lumen 110 allows for a medical device such as a lead or catheter to be advanced through the assembly 103. The valve housing 104 has spaced apart wings 112 and 114.

The proximal sheath section 102B includes perforations 166 which help propagate splitting of the sheath 102. Once the lead or catheter is properly positioned, the valve introducer assembly 100 is split apart for removal from the vasculature. The valve housing 104 including the valve membrane 106 are then separated along score lines 138 running along the lower valve body 104A and the valve cap 104B.

Helgeson et. al. U.S. Pat. No. 8,043,263 incorporated herein by reference, is directed to a splittable delivery device assembly for the delivery of a cardiac surgical device. The delivery or insertion tool also includes a hemostasis valve, both of which are splittable or could be removed after the cardiac surgical device of interest is introduced. Beginning with FIG. 9 et. seq. in Helgeson, the delivery device 10 may be a catheter or sheath 10 with shaft 20 including a lumen extending the length of the device 10 providing a passageway for a surgical device 5 to enter the body. After the device 5 has been introduced, the delivery device 10 is removed by means of slitting along its length. A hemostasis valve 25 may be integrated into the hub 15 and may be removed by slitting or a cap 30 may be used to retain the valve 25 within the hub 15. There is a transvalvular insertion tool 305 beginning in FIG. 9.

Nardeo et. al., U.S. Pre-Grant Publication 2010/0292646 incorporated herein by reference, is directed to a tear away sheath assembly with split hemostasis valve seal. The assembly 100 has a splittable sheath 102 along with a splittable hub 110 and a split valve 150, 250. The valve 150, 250 is formed with two separate halves 152A and 152B that are fused or adhered to each other to form a sealed weak bond 154 that is broken during the splitting of the sheath. The tool may be used to insert a catheter as part of a medical procedure and once in place, the introducer along with the valve may be removed.

Fisher et. al., U.S. Pat. No. 8,105,287 incorporated herein by reference, is directed to a tear away introducer sheath with hemostasis valve. Referring the figures, the catheter introducer sheath assembly 100 comprises a sheath portion 132 with a tear seam defined along the length of sheath 132 and indicated by score lines 130. Score lines 130 allow the sheath portion 132 to be split apart after the catheter (not shown) has been inserted and in place. The sheath hub portion 140 consists of two half-portions 142 and affixed to the sheath portions on each side of the tear seam 130. The sheath hub 140 may include a valve 300 and is sectioned along a plane perpendicular to the plane of separation of the sheath hub as seen in FIGS. 4 and 5 of Fisher.

Potter et. al., U.S. Pat. No. 7,985,232 incorporated herein by reference, is directed to a detachable hemostasis valve and splittable sheath assembly and provides an alternative to the systems described above but intended to accomplish the same ends. The hemostasis valve is connected to a splittable sheath and when the handles are pulled to split the sheath, the valve is disengaged from the sheath assembly. The coupling system for the hemostasis device 70 and the splittable sheath 100 is seen in FIGS. 1-4 of Potter and includes a valve housing 90. A tool provides for the introduction of a lead or guide wire as well as a dilator or catheter to be inserted into the hemostasis device 70 and splittable sheath 100 for delivery to a particular site in a medical procedure.

Johnson et. al. U.S. Pat. No. 8,105,315 incorporated herein by reference, is noted for showing an example of a bypass tool, called an actuator 50.

The prior art designs for splittable valved introducers do not address the problems arising from friction between the valve and intravascular tools, particularly pliable or soft intravascular instruments. What is needed is a universal TVI tool, which is configured to be inserted into a valved introducer to open the hemostasis valve of the introducer and allow an unobstructed passage for a delicate pacing or defibrillator lead or other instrument.

### BRIEF SUMMARY

The illustrated embodiments are directed to a transvalvular insertion tool for providing a passage through a hemostatically valved introducer. The tool includes at least one tab, a hub coupled to the tab, a longitudinally rigid sheath for insertion through the valved introducer to provide an obstruction free path for delivery of a lead or vascular device through the valved introducer, and means for separating the hub and rigid sheath to allow removal from the lead or device without removal of the transvalvular insertion tool over either end of the lead or vascular device.

The means for separating includes longitudinal halves of the rigid sheath and a temporary mechanical coupling for selectively retaining the longitudinal halves together.

In one embodiment the means for separating includes a longitudinal line of separation of the rigid sheath and a clam-shell mechanism for spreading the longitudinal line of separation of the rigid sheath apart to selectively create a longitudinal gap in the rigid sheath.

In another embodiment the means for separating includes a longitudinal line of weakness of the rigid sheath. The rigid sheath includes an external stop positioned between the hub and distal end of the sheath.

The illustrated embodiments of the invention are directed to a universal transvalvular insertion tool for providing a passage through a multiplicity of differently designed hemostatically valved introducers for a lead or vascular device, each valved introducer having a valve within a valved body. The tool includes a longitudinally rigid sheath having a lumen for insertion through the valve of any one of the multiplicity of differently designed hemostatically valved introducers to provide a substantially obstruction free path for delivery of a lead or vascular device through the valve and valved body of each of the valved introducers. The rigid sheath has a length selected to extend through the valve without substantial deformation of the lumen of the rigid sheath. Means for separating the rigid sheath allows removal from the lead or device without removal of the transvalvular insertion tool over either end of the lead or vascular device.

The tool further includes a hemostatic introducer to provide a kit.

In one embodiment the sheath and/or hub separate into two equal halves.

In one embodiment the hub and/or sheath includes a living hinge that separates into two connected equal halves. A living hinge is a thin flexible hinge (flexure bearing) made from the same material as the two rigid pieces it connects, rather than cloth, leather, or some other flexible substance. It is typically thinned or cut to allow the rigid pieces to bend along the line of the hinge.

In one embodiment the hub and/or sheath is arranged and configured to be separated and put back together with or without an intravascular tool being inserted therethrough at the time of recombination for indefinite repeated usage on each instance of application if desired.

In one embodiment the hub and/or sheath has a plurality of interlocking angled projections along the sheath to prevent the sheath halves from becoming misaligned from forces exerted thereon by the valved introducers.

The sheath and/or hub includes a sealing member or gasket between the halves of sheath and/or hub that seals the halves to each other when assembled, which sealing member or gasket is softer than the halves of the sheath and/or hub.

The sheath need only be rigid to the extent that the sheath will not deform when the distal end of the sheath stops against the inside of the introducer valve housing.

The tool is arranged and configured to be used in conjunction with any valved introducer to provide a separable unobstructed conduit through the valve.

The rigid sheath is arranged and configured to bottom out on the valved body, which allows a user to be instructed or directed to push the tool through the valve until it stops.

The means for separating the rigid sheath and/or hub to allow removal from the lead or device without removal of the transvalvular insertion tool over either end of the lead or vascular device further includes means for allowing reassembly of the transvalvular insertion tool.

In one embodiment the sheath and/or hub has two opposing portions and the means for separating and allowing reassembly includes fingers with an interference fit into a mating indentation in the opposing hub sheath and/or sheath portions.

In one embodiment the hub has two opposing portions and the means for separating and allowing reassembly includes a snap peg and hole combination for coupling the two sheath and/or hub portions together.

In one embodiment the means for separating and allowing reassembly includes a hinged clam shell mechanism having tabs and an opening on an exterior surface of the hub opposing the tabs of the clam shell mechanism.

In one embodiment the means for separating and allowing reassembly includes an insert disposed in the sheath and/or hub and a tongue and groove combination defined at least in part in the insert disposed in the sheath and/or hub.

In one embodiment the means for separating and allowing reassembly includes a single split tab with an opposing resilient hub portion having a longitudinal slit and sheath defined inwardly at least in the hub between the split portions of the tab to provide a clam shell mechanism for opening the hub.

In one embodiment the means for separating and allowing reassembly includes a plurality of snap interlaced fingers and recesses longitudinally defined in opposing portions of the sheath, and a tenon and mortise combination defined in an inner surface of the opposing hub portions.

In one embodiment the means for separating and allowing reassembly includes a single tab and an opposing split cylinder portion forming the hub and sheath.

In one embodiment the means for separating and allowing reassembly includes a one-way swing latch combination defined in an inner portion of the sheath and/or hub portions.

In one embodiment the means for separating and allowing reassembly includes a resealable membrane combination disposed longitudinally along the hub and/or sheath.

In one embodiment the means for separating and allowing reassembly includes a single tab having a half cylinder defined therein and an opposing half cylinder to form the hub and sheath and with a hinge on one side of the opposing half cylinder coupled to the tab and with a split defined opposing the hinge and extending longitudinally along the sheath.

In one embodiment the means for separating and allowing reassembly includes a slidable, longitudinal tongue and groove combination defined in the hub and/or sheath.

In one embodiment the means for separating and allowing reassembly includes a single tab and an opposing cylinder forming the hub and sheath with an open slot longitudinally defined therein and extending along the hub and sheath.

In one embodiment the means for separating and allowing reassembly includes opposing portions of the hub having opposing sides and a snap finger and recess combination defined in the opposing sides of the sheath and/or hub.

The illustrated embodiments also include a method of using a transvalvular insertion tool for providing a passage through a hemostatically valved introducer comprising the steps of inserting a longitudinally rigid sheath through the valved introducer to provide an obstruction free path for delivery of a lead or vascular device through the valved introducer, and separating the hub and rigid sheath to allow removal from the lead or device without removal of the transvalvular insertion tool over either end of the lead or vascular device.

In one embodiment the step of inserting a longitudinally rigid sheath through the valved introducer is performed until the tool is stopped in the valved introducer.

In one embodiment the method further includes reassembling the hub and sheath after use for a repeated use.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a perspective view of two prior art TVI tools.

FIG. 2a is a perspective view of one half of an snap-together embodiment of the invention, and FIG. 2b is a perspective view of two halves of the type shown in FIG. 2a combined to form a completed assembly.

FIG. 3 is a perspective view of a one piece embodiment.

FIG. 4a is a perspective view of a clam-shell embodiment shown in the closed configuration.

FIG. 4b is a perspective view of a dam-shell embodiment shown in the open configuration.

FIG. 5a is a perspective view in enlarged scale of the snap-together embodiment of FIG. 2a.

FIG. 5b is a perspective view in enlarged scale of the snap-together embodiment of FIG. 5a where the second half is combined to provide the complete assembly.

FIG. 6a is a side cross-sectional exploded view of a snap peg-and-hole embodiment.

FIG. 6b is a top plan exploded view of the embodiment of FIG. 6a.

FIG. 6c is a side plan exploded view of the embodiment of FIG. 6a.

FIG. 6d is a perspective exploded view of the embodiment of FIG. 6a.

FIG. 6e is end plan view of the embodiment of FIG. 6a.

FIG. 7a is a side cross-sectional unexploded view of a snap peg-and-hole embodiment.

FIG. 7b is a top plan unexploded view of the embodiment of FIG. 7a.

FIG. 7c is a side plan unexploded view of the embodiment of FIG. 7a.

FIG. 7d is a perspective unexploded view of the embodiment of FIG. 7a.

FIG. 7e is end plan view of a peg-and-hole embodiment of FIG. 7a.

FIG. 8a is a top plan view of the embodiment of FIGS. 4a and 4b in a dosed configuration.

FIG. 8b is a side plan view of the embodiment of FIGS. 4a and 4b in a closed configuration.

FIG. 8c is a perspective view of the embodiment of FIGS. 4a and 4b in a closed configuration.

FIG. 8d is a side plan view of the embodiment of FIGS. 4a and 4b in a closed configuration.

FIG. 9a is a top plan view of the embodiment of FIGS. 4a and 4b in an opened configuration.

FIG. 9b is a side plan view of the embodiment of FIGS. 4a and 4b in an opened configuration.

FIG. 9c is a perspective view embodiment of FIGS. 4a and 4b in an opened configuration.

FIG. 9d is a side plan view embodiment of FIGS. 4a and 4b in an opened configuration.

FIG. 10a is a top plan view of an embodiment of the invention with a longitudinal tongue and groove defined in the hub shown in the separated configuration.

FIG. 10b is a side plan view of the embodiment of the invention of FIG. 10a.

FIG. 10c is a perspective view of the embodiment of the invention of FIG. 10a.

FIG. 10d is a side plan view of an embodiment of the invention of FIG. 10a.

FIG. 10e is an enlarged perspective view of the insert regional 10-10 of FIG. 10c.

FIG. 11a is a top plan view of an embodiment of the invention with a tongue and groove structure defined in inserts in the hub shown in the dosed configuration.

FIG. 11b is a side plan view of the embodiment of the invention of FIG. 11a.

FIG. 11c is a perspective view of an embodiment of the invention of FIG. 1a.

FIG. 11d is a side plan view of an embodiment of the invention of FIG. 1a.

FIG. 11e is an enlarged perspective top plan view of the insert region 11-11 of FIG. 11c.

FIG. 12a is a top plan view of an embodiment of the invention with a single split tab extending from a resilient cylindrical hinge defining one side of the hub shown in the dosed configuration.

FIG. 12b is a side plan view of the embodiment of the invention of FIG. 12a.

FIG. 12c is a perspective view of an embodiment of the invention of FIG. 12a.

FIG. 12d is a side plan view of an embodiment of the invention of FIG. 12a.

FIG. 13a is a top plan view of an embodiment of the invention with a single split tab extending from a resilient cylindrical hinge defining one side of the hub shown in an opened configuration.

FIG. 13b is a side plan view of the embodiment of the invention of FIG. 13a.

FIG. 13c is a perspective view of an embodiment of the invention of FIG. 13a.

FIG. 13d is a side plan view of an embodiment of the invention of FIG. 13a.

FIG. 14a is a top plan view of an embodiment of the invention with a plurality of interlaced snap-fit fingers or angled projections defined in the hub and down the longitudinal axis of the sheath shown in the closed configuration.

FIG. 14b is a side plan view of the embodiment of the invention of FIG. 14a.

FIG. 14c is a perspective view of the embodiment of the invention of FIG. 14a.

FIG. 14d is a side plan view of the embodiment of the invention of FIG. 14a.

FIG. 15a is a top plan view of one half of the hub and sheath of the embodiment of the invention of FIGS. 14a-14d with a plurality of snap-fit fingers defined in the hub and down the longitudinal axis of the sheath.

FIG. 15b is a side plan view of one half of the hub and sheath of the embodiment of the invention of FIG. 15a.

FIG. 15c is a perspective view of one half of the hub and sheath of the embodiment of the invention of FIG. 15a.

FIG. 15d is a side plan view of one half of the hub and sheath of the embodiment of the invention of FIG. 15a.

FIG. 16a is a perspective view of the embodiments of FIGS. 15a-16d shown in an exploded or fully separated view.

FIG. 16b is an enlarged view of the portion of the embodiment in FIG. 16a within insert region B.

FIG. 16c is a perspective view of the embodiments of FIGS. 15a-16d shown in a view where the portions have just begun to become engage or disengaged from each other.

FIG. 16d is an enlarged view of the portion of the embodiment in FIG. 16c within insert region C.

FIG. 16e is a perspective view of the embodiments of FIGS. 15a-16d shown in a fully assembled view.

FIG. 16f is an enlarged view of the portion of the embodiment in FIG. 16e within insert region D.

FIG. 17a is a top plan view of an embodiment of the invention with a single tab extending from a separable cylinder defining one side of the hub shown in the closed configuration.

FIG. 17b is a side plan view of the embodiment of the invention of FIG. 17a.

FIG. 17c is a perspective view of an embodiment of the invention of FIG. 17a.

FIG. 17d is a side plan view of an embodiment of the invention of FIG. 17a.

FIG. 18a is a top plan view of an embodiment of the invention with a single tab extending from a separable cylinder defining one side of the hub shown with the cylinder separated along its longitudinal length.

FIG. 18b is a side plan view of the embodiment of the invention of FIG. 18a.

FIG. 18c is a perspective view of an embodiment of the invention of FIG. 18a.

FIG. 18d is a side plan view of an embodiment of the invention of FIG. 18a.

FIG. 19a is a side cross sectional view of an embodiment of the invention with a one way swing latch defined in the hub shown in a fully separated configuration.

FIG. 19b is a top plan view of an embodiment of the invention of FIG. 19a.

FIG. 19c is a side plan view of the embodiment of the invention of FIG. 19a.

FIG. 19d is a perspective view of an embodiment of the invention of FIG. 19a.

FIG. 19e is a side plan view of an embodiment of the invention of FIG. 19a.

FIG. 20a is a side cross sectional view of an embodiment of the invention with a one way swing latch defined in the hub shown in a partially engaged configuration.

FIG. 20b is a top plan view of an embodiment of the invention of FIG. 20a.

FIG. 20c is a side plan view of the embodiment of the invention of FIG. 20a.

FIG. 20d is a perspective view of an embodiment of the invention of FIG. 20a.

FIG. 20e is a side plan view of an embodiment of the invention of FIG. 20a.

FIG. 21a is a side cross sectional view of an embodiment of the invention with a one way swing latch defined in the hub shown in a fully assembled configuration.

FIG. 21b is a top plan view of an embodiment of the invention of FIG. 21a.

FIG. 21c is a side plan view of the embodiment of the invention of FIG. 21a.

FIG. 21d is a perspective view of an embodiment of the invention of FIG. 21a.

FIG. 21e is a side plan view of an embodiment of the invention of FIG. 21a.

FIG. 22a is a top plan view of an embodiment of the invention with a longitudinal sealing or adhesive membrane defined through the hub and sheath shown in a fully unsealed configuration.

FIG. 22b is a side plan view of the embodiment of the invention of FIG. 22a.

FIG. 22c is a perspective view of an embodiment of the invention of FIG. 22a.

FIG. 22d is a side plan view of an embodiment of the invention of FIG. 22a.

FIG. 23a is a top plan view of an embodiment of the invention with a longitudinal sealing or adhesive membrane defined through the hub and sheath shown in a fully sealed configuration.

FIG. 23b is a side plan view of the embodiment of the invention of FIG. 23a.

FIG. 23c is a perspective view of an embodiment of the invention of FIG. 23a.

FIG. 23d is a side plan view of an embodiment of the invention of FIG. 23a.

FIG. 24a is a top plan view of an embodiment of the invention with a single tab extending from a cylinder defining one side of the hub, which cylinder is separable on one side edge of the cylinder or both shown in an attached configuration.

FIG. 24b is a side plan view of the embodiment of the invention of FIG. 24a.

FIG. 24c is a perspective view of an embodiment of the invention of FIG. 24a.

FIG. 24d is a side plan view of an embodiment of the invention of FIG. 24a.

FIG. 25a is a top plan view of an embodiment of the invention with a single tab extending from a cylinder defining one side of the hub, which cylinder is separable on one side edge of the cylinder or both shown in a detached configuration.

FIG. 25b is a side plan view of the embodiment of the invention of FIG. 25a.

FIG. 25c is a perspective view of an embodiment of the invention of FIG. 25a.

FIG. 25d is a side plan view of an embodiment of the invention of FIG. 25a.

FIG. 26a is a top plan view of an embodiment of the invention with a tongue and groove attachment longitudinally defined through the hub and sheath in which the two opposing portions longitudinally slide together and apart from each other and shown in a detached configuration.

FIG. 26b is a side plan view of the embodiment of the invention of FIG. 26a.

FIG. 26c is a perspective view of an embodiment of the invention of FIG. 26a.

FIG. 26d is a side plan view of an embodiment of the invention of FIG. 26a.

FIG. 26e is a top plan view in enlarged scale of the insert region A of FIG. 26a.

FIG. 27a is a top plan view of an embodiment of the invention with a tongue and groove attachment longitudinally defined through the hub and sheath in which the two opposing portions longitudinally slide together and apart from each other and shown in a partially detached or partially assembled configuration.

FIG. 27b is a side plan view of the embodiment of the invention of FIG. 27a.

FIG. 27c is a perspective view of an embodiment of the invention of FIG. 27a.

FIG. 27d is a side plan view of an embodiment of the invention of FIG. 27a.

FIG. 27e is a top plan view in enlarged scale of the insert region A of FIG. 27a.

FIG. 28a is a top plan view of an embodiment of the invention with a tongue and groove attachment longitudinally defined through the hub and sheath in which the two opposing portions longitudinally slide together and apart from each other and shown in a fully assembled configuration.

FIG. 28b is a side plan view of the embodiment of the invention of FIG. 28a.

FIG. 28c is a perspective view of an embodiment of the invention of FIG. 28a.

FIG. 28d is a side plan view of an embodiment of the invention of FIG. 28a.

FIG. 28e is a top plan view in enlarged scale of the insert region A of FIG. 28a.

FIG. 29a is a top plan view of an embodiment of the invention with a single tab wherein an open slot is defined an opposing cylinder portion of the hub and continues longitudinally along the longitudinal axis of the sheath.

FIG. 29b is a side plan view of the embodiment of the invention of FIG. 29a.

FIG. 29c is a perspective view of an embodiment of the invention of FIG. 29a.

FIG. 29d is a side plan view of an embodiment of the invention of FIG. 29a.

FIG. 30a is a top plan view of an embodiment of the invention with an opposing snap finger and mating recess is defined in the mid portion of the hub and shown in a separated configuration.

FIG. 30b is a side plan view of the embodiment of the invention of FIG. 30a.

FIG. 30c is a perspective view of an embodiment of the invention of FIG. 30a.

FIG. 30d is a side plan view of an embodiment of the invention of FIG. 30a.

FIG. 31a is a top plan view of an embodiment of the invention with an opposing snap finger and mating recess is defined in the mid portion of the hub and shown in a fully assembled configuration.

FIG. 31b is a side plan view of the embodiment of the invention of FIG. 31a.

FIG. 31c is a perspective view of an embodiment of the invention of FIG. 31a.

FIG. 31d is a side plan view of an embodiment of the invention of FIG. 31a.

FIG. 32a is a top plan view of an embodiment of the invention with an opposing line of weakness defined in the mid portion of the hub and shown in a fully assembled configuration.

FIG. 32b is a side plan view of the embodiment of the invention of FIG. 32a.

FIG. 32c is a perspective view of an embodiment of the invention of FIG. 32a.

FIG. 32d is a side plan view of an embodiment of the invention of FIG. 32a.

FIG. 32e is a cross sectional view of the embodiment of FIG. 32a.

FIG. 33a is a top plan view of an embodiment of the invention with an opposing line of weakness defined in the mid portion of the hub and shown in a fully assembled configuration.

FIG. 33b is a side plan view of the embodiment of the invention of FIG. 33a.

FIG. 33c is a perspective view of an embodiment of the invention of FIG. 33a.

FIG. 33d is a side plan view of an embodiment of the invention of FIG. 33a.

FIG. 33e is a cross sectional view of the embodiment of FIG. 33a.

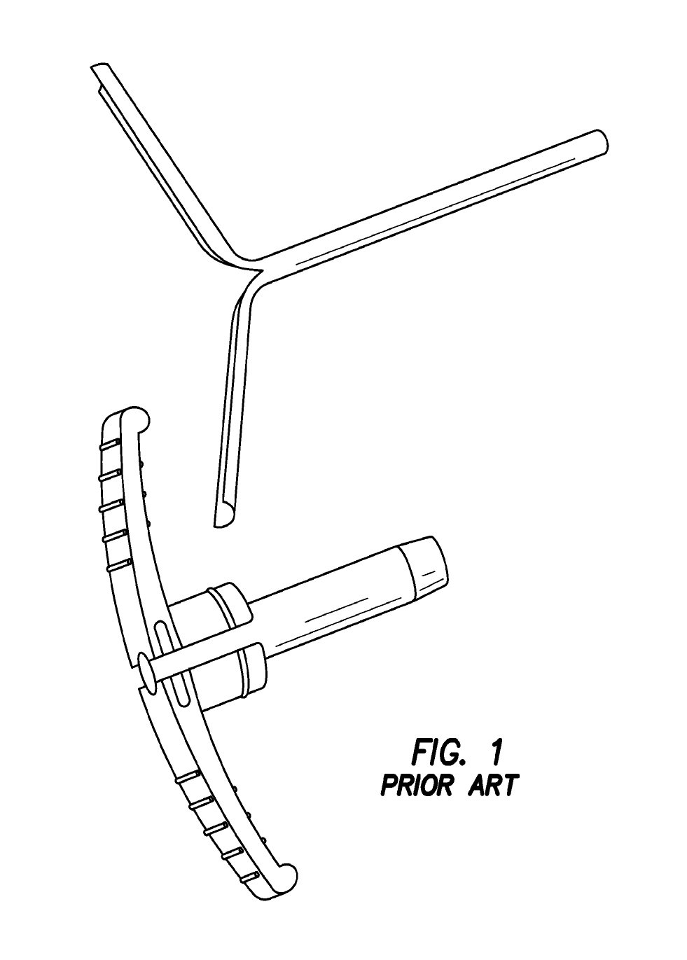

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

One embodiment of the invention is a transvalvular insertion tool (TVI tool) 10 is characterized by a rigid construction of the bypass sheath 12 that will not collapse when it is pushed up against the introducer housing, yet is still able to be separated from the lead after being used. The need for the rigid construction is to allow the TVI tool 10 to work with all existing hemostasis valved introducers where the distal tubular end 14 of the TVI tool 10 needs to be long enough to completely extend through some hemostasis valve introducers yet will “bottom out” on the housing of a shorter introducer, whereby the rigid construction prevents the distal tubular end from collapsing.

The TVI tool can be fabricated in numerous ways including machining, molding, casting, or forging and can be comprised of one or more materials including metal, plastic, ceramic, composite, rubber or elastomer, but preferably from a rigid plastic such as acetal, polycarbonate, polyester, polypropylene, polyurethane, nylon, PEBA, or ABS.

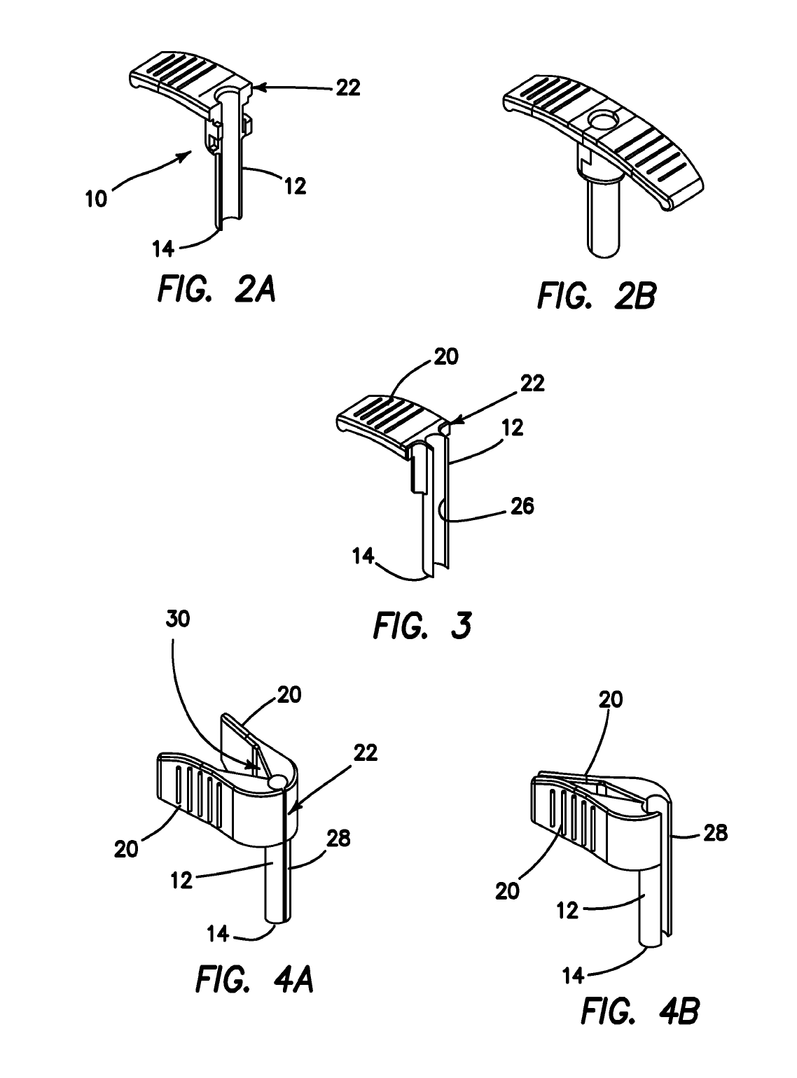

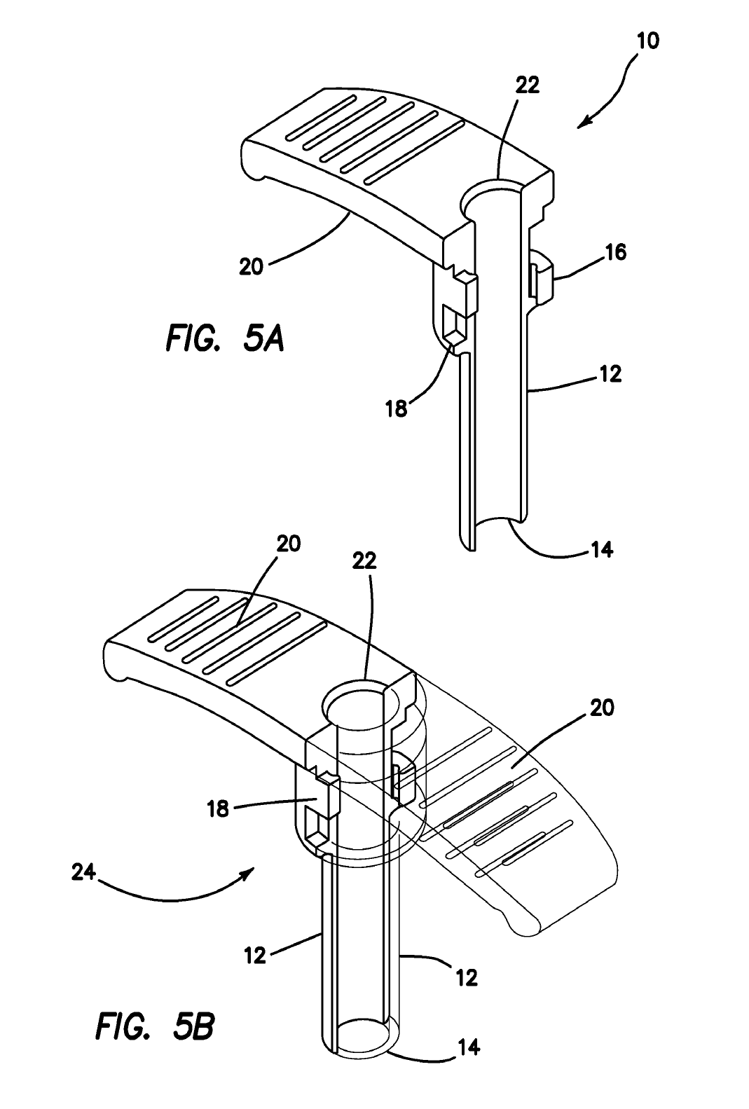

FIGS. 2a and 2b are perspective views of an embodiment, which is a two-piece, snap-together design. Hub 22 is fabricated in two halves and has a pair of radially extending fingers or male snap moldings 16 on the upper portion of one half of hub 22. Each half hub 22 includes a horizontally oriented tab 20 radially extending from the upper portion of half hub 22. The embodiment of FIGS. 2a and 2b is shown in enlarged scale in FIGS. 5a and 5b. The bypass sheath 12 is provided with an integrally molded tab 20 on its proximal hub end 22, a male snap molding 16 on one side of the proximal portion of sheath 12 and a female snap molding 18 on opposing side of the proximal portion of sheath 12. An identical TVI tool half is provided and mated with the first half to form a combined assembly 24 shown in FIG. 5b where the second half is depicted in phantom shadow outline. The lower portion of the same half hub 22 had defined therein receiving cavities or female snap moldings 18 into which corresponding male snap moldings 16 from the opposing half hub 22 are disposed. Detent protrusions may be defined in female snap moldings 18 over which male snap moldings 16 ride and then snap into place once the detent protrusions are cleared. The matching and opposing half of hub 22 has a mating arrangement, namely male snap moldings 16 extending radially from the lower portion of half hub 22 with female snap moldings (not shown) defined into the upper portion of half hub 22 for receipt of male snap moldings 16 from the opposing half hub 22.

The other embodiments include a slotted embodiment shown in perspective view in FIG. 3, and a clamshell/clothespin design shown in perspective view in FIGS. 4a and 4b. The slotted embodiment of FIG. 3 is formed as an integrally molded half with a longitudinal slot 26 defined along sheath 12 of the half provided. The slotted embodiment has a single horizontally oriented tab 20 integrally formed with a half hub 22 and with half cylindrical sheath 12. The width of the slot 26, i.e. the extent to which a full cylindrical enclosure is not provided can be varied according to the application at hand, namely the maximal diameter of the transvascular tool to be accommodated by TVI tool 10.

The clam shell embodiment of FIGS. 4a and 4b includes a rigid sheath 12, but is composed of a resilient material which can be torqued open and dosed or at least includes a longitudinal living hinge 30 of resilient material or weakening opposite slit 28, which allows it to be opened and closed as vertically oriented tabs 20 are squeezed together as shown in FIG. 4b to open slit 28 or allowed to separate under the resilience of sheath 12 and hinge 30 as shown in FIG. 4a. The sheath 12 of the TVI tool 10 in FIGS. 4a and 4b is stiff and will not collapse, distort or bend when abutted against an obstruction in the valve body, introducer or the transition between them.

The rigid or at least longitudinally stiff TVI tool 10 of any of the embodiments can be forced through any obstruction or at least forced up against an obstruction to allow a delicate lead to pass through without a friction fit or interference with the TVI tool 10 or from valve membrane of the hemostatic introducer. In all embodiments the TVI tool 10 can be separated to open, or into at least two parts, or otherwise manipulated to allow it to be taken off the pacemaker lead or intravascular device without being slid off the proximal end of the lead or device, which is impossible in any case since there is a large electrical connector or other obstruction at the proximal end of the tool or lead.

Note that one preferred embodiment of the universal TVI tool 10 does not peel like a conventional splittable/peel away sheath, along lines of weakness or molecular oriented material of sheath 12, which cannot be rejoined and reused. Nor is the conventional TVI tool, which is sliced open, available for reassembly or reuse. Rather the TVI tool 10 of the illustrated embodiments separates into two portions or has a structure so that it can be reassembled, if necessary or desired, and reused. The Universal TVI tool 10 may be separated and put back together or rejoined with or without an intravascular tool being inserted therethrough at the time of recombination for indefinite re-sterilization and repeated usage on each instance of application if desired.

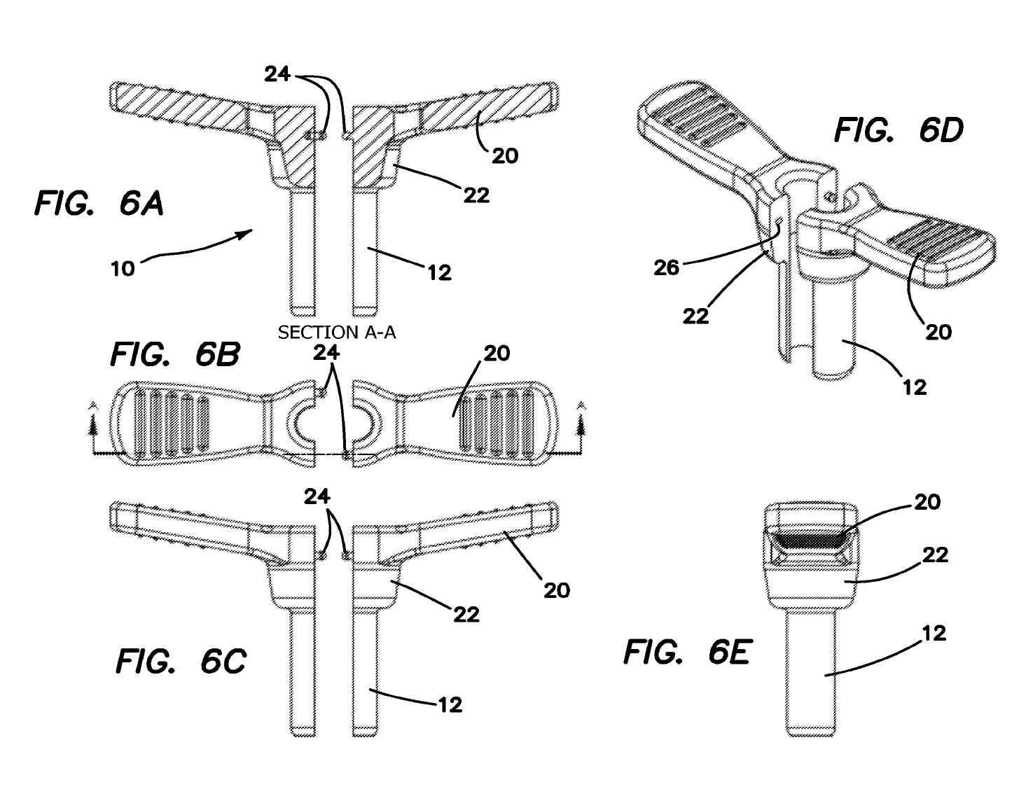

Many embodiments may be devised to allow the intravascular tool to be inserted therethrough at the time of recombination for indefinite repeated usage on each instance of application. FIGS. 2a-4b illustrate three embodiments, which are shown in greater detail along with a plurality of additional embodiments in FIGS. 6a-32d. A plurality of different coupling mechanisms or schemes are illustrated in these embodiments. It is to be understood that although the illustrated embodiment may show the mechanism or scheme used to join and separate the hub 22, in many cases the same or similar mechanism or scheme can be extended to the sheath 12, if desired, and vice versa. This collection of illustrated embodiments does not exhaust the number of possible mechanisms for providing for the TVI tool 10 of the invention, which may be realized in many other ways without departing from the spirit and scope of the invention.

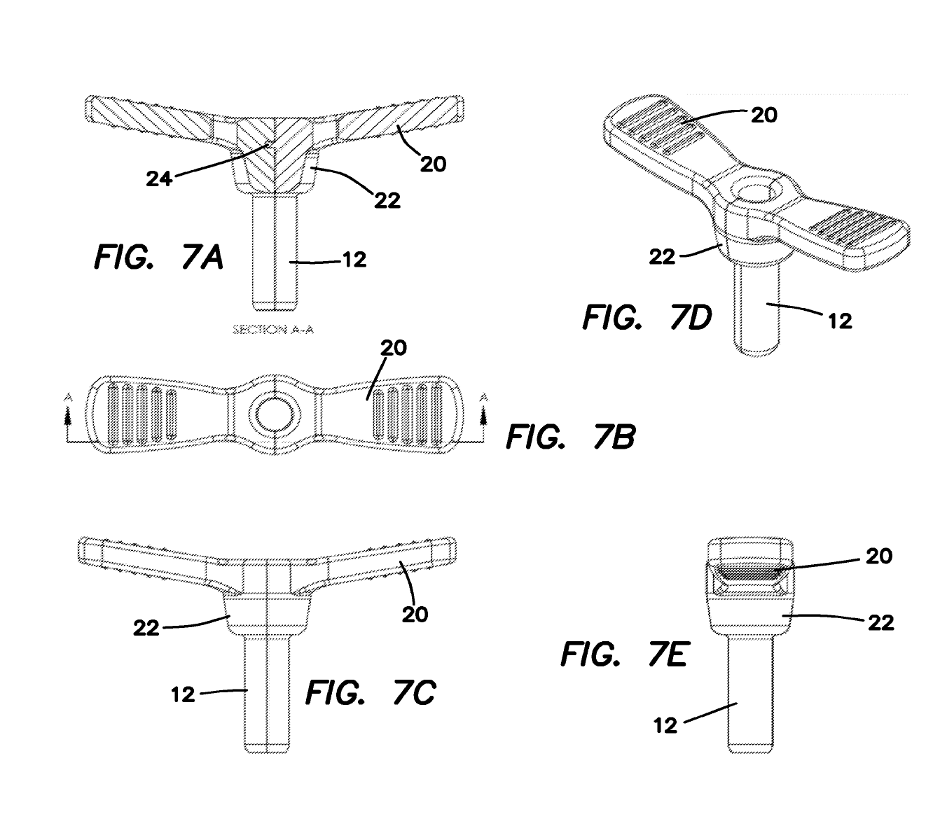

For example FIG. 6a is a side cross-sectional exploded view of a snap peg-and-hole embodiment with FIG. 6b a top plan exploded view, FIG. 6c a side plan exploded view and FIG. 6d is a perspective exploded view. The views of FIGS. 6a-6e are views in which the two halves of TVI 10 are shown in the separated configuration. Hub 22 of TVI 10 is formed to two opposing portions or halves 22 with a mating peg 24 provided on one side and on the opposing side of the same half or portion is a mating hole 26, best seen in FIG. 6d, into which peg 28 snaps or can be reversibly force fit. Peg 28 is provided with an slightly expanded distal end or head 29 to provide for a compression fit into hole 26. The proximal end of sheath 12 is coupled to the bottom of corresponding portions of hub 22 and is formed into two longitudinal halves or portions which freely or flushly join or meet with each other. FIGS. 7a-7d show the portions in an assembled configuration.

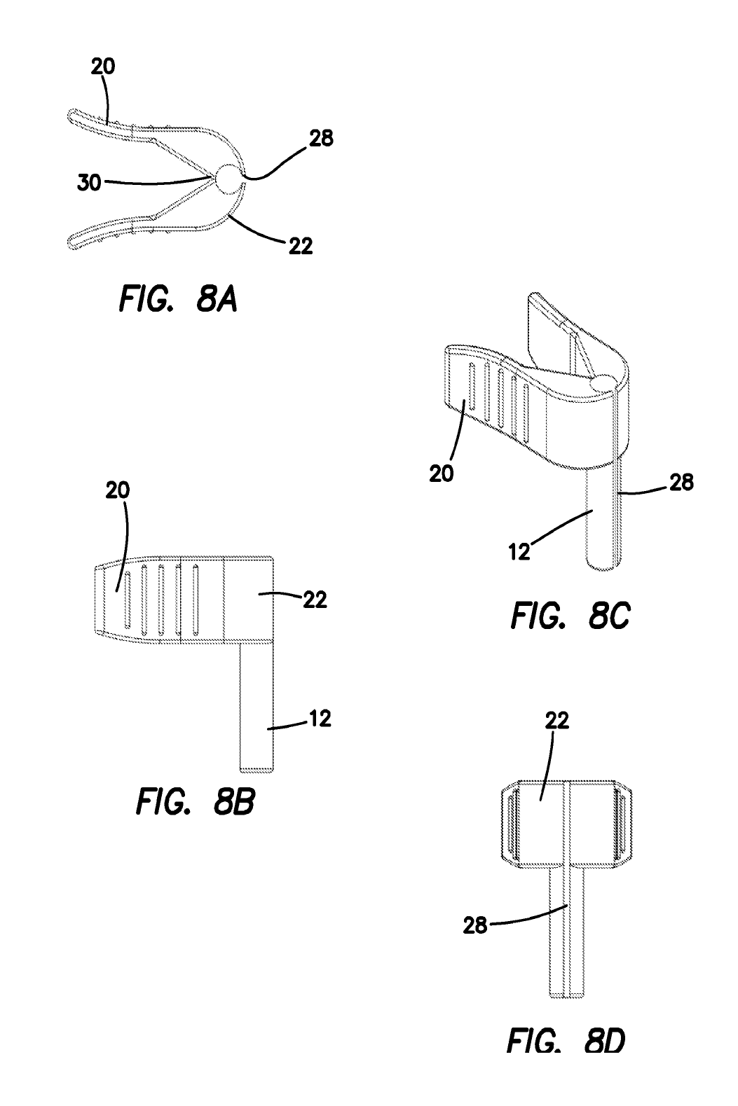

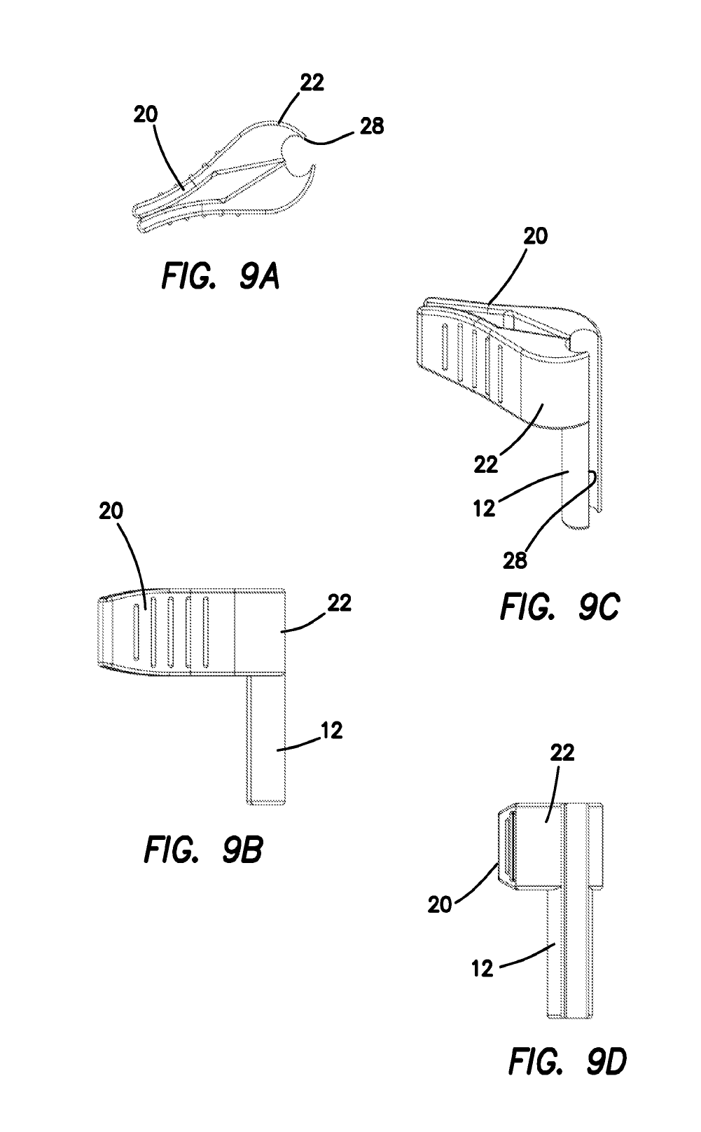

The clam shell embodiment of FIGS. 4a and 4b are shown in more detail in the closed and open configurations in FIGS. 8a-8d and FIGS. 9a-9d respectively. In the embodiment of FIGS. 9a-9d, the tabs 20 provide a surface which is oriented in a direction generally parallel to the axis of hub 22 and sheath 12 as opposed to the generally perpendicular surfaces presented by the embodiment in FIGS. 6a-7e for example. Hub 22 is thus integral with tabs 20 and shares a continuous surface with tabs 20.

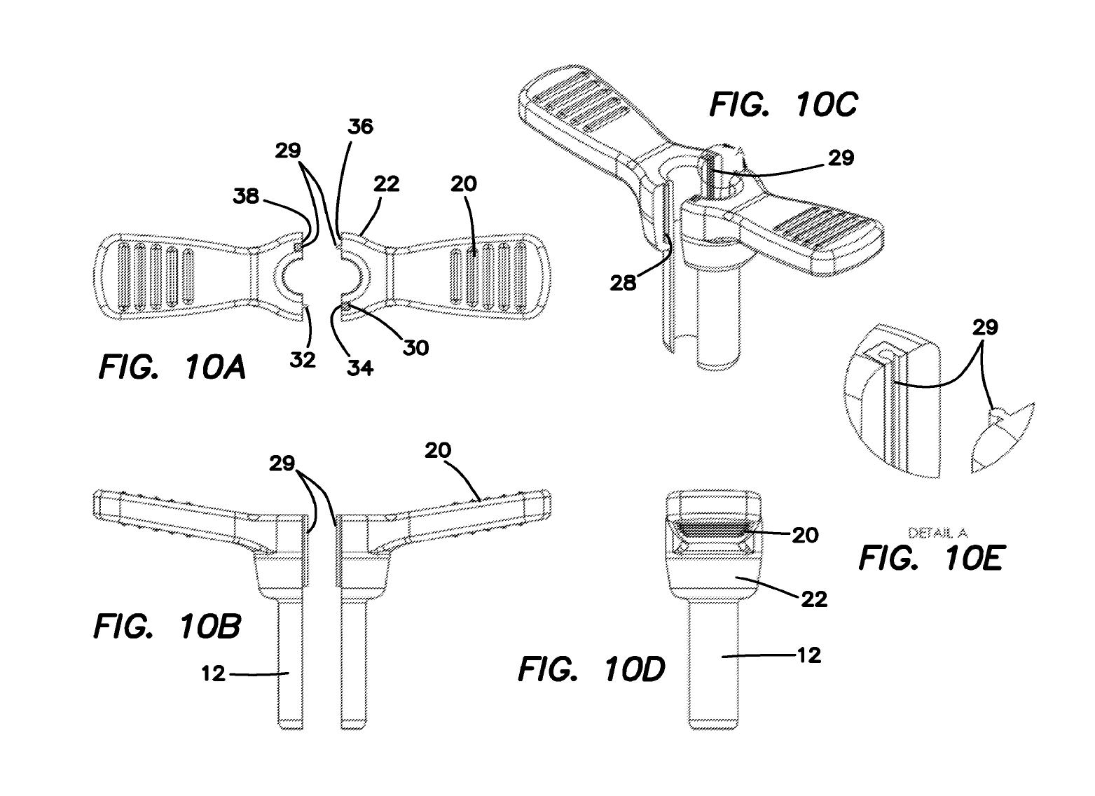

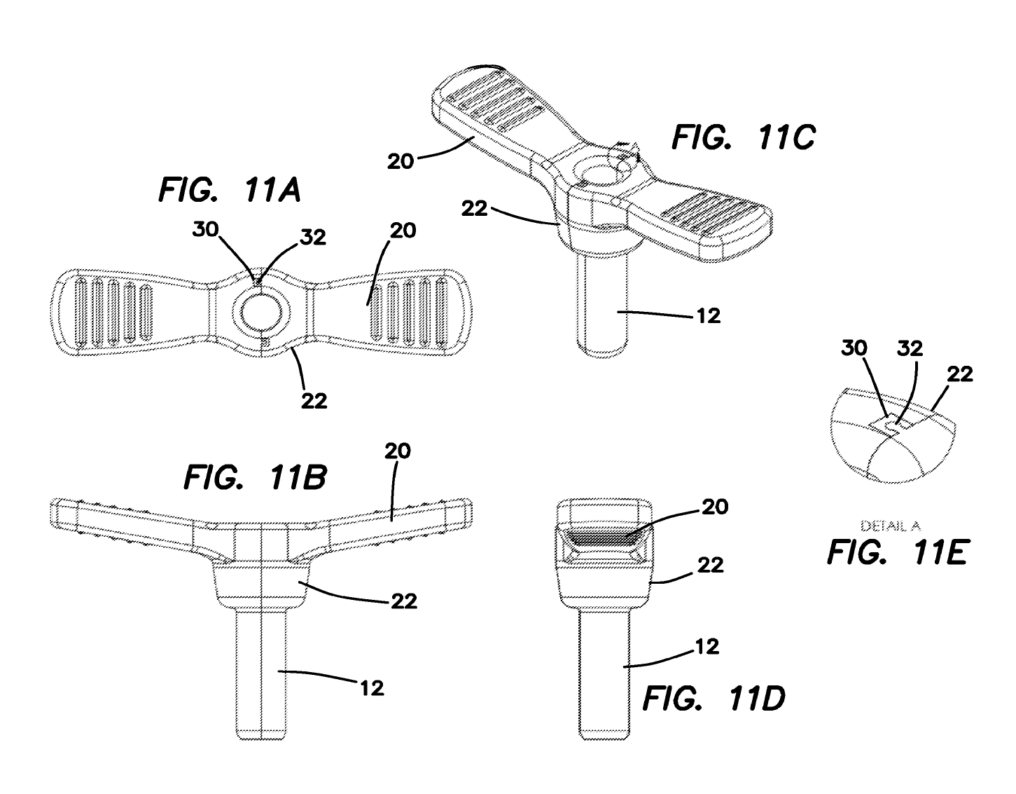

FIGS. 10a-10e show an embodiment with a longitudinal tongue and groove combination 31 defined in the hub 22 in an open configuration and FIGS. 11a-11e in a closed configuration. A tongue 32 is provided longitudinally down one side of the inner surface 36 of hub 22. On the opposing surface 38 of the opposing hub portion is a mating groove 34 defined in a longitudinal insert 33 disposed into hub 22. Insert 33 may be a selected resilient or softer material than that used for hub 22 to provide a smooth and positive snap or clasping attachment for the combination 31. Again the proximal end of sheath 12 is coupled to the bottom of corresponding portions of hub 22 and is formed into two longitudinal halves or portions which freely or flushly join or meet with each other. Sheath 12 mates freely and flushly together along a longitudinal access.

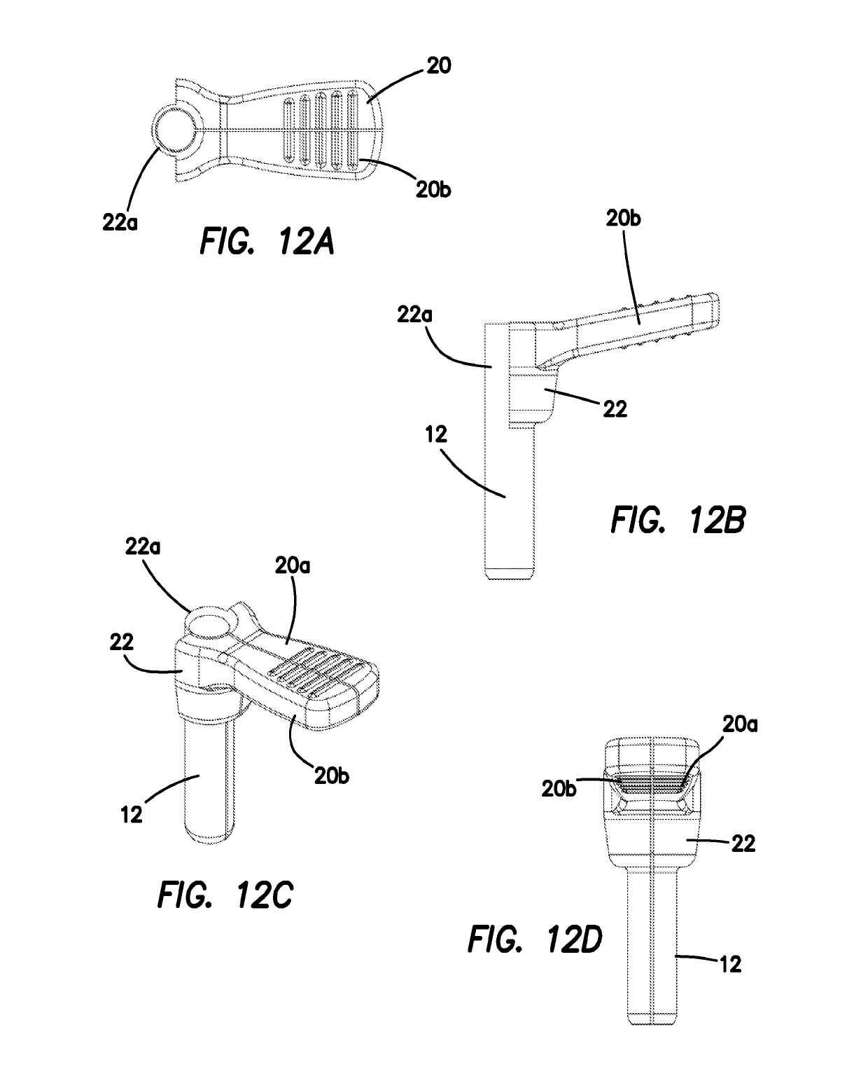

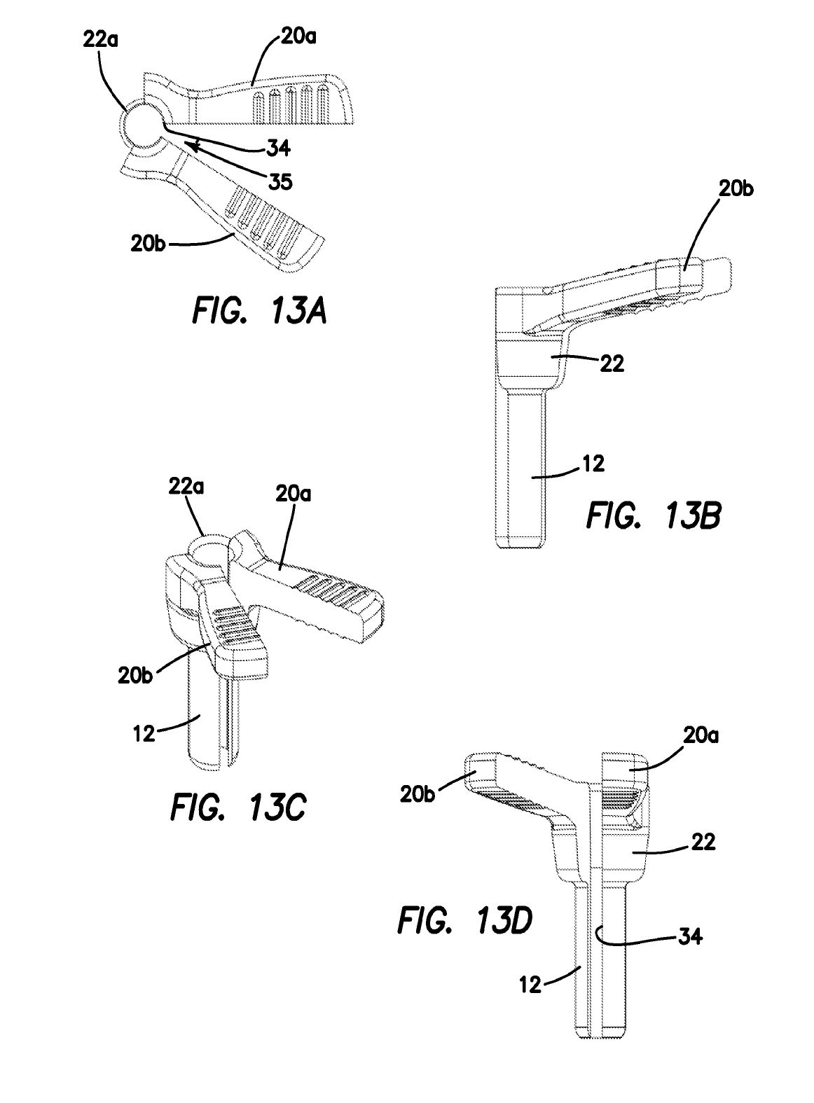

FIGS. 12a-12d show an embodiment of the invention with a single horizontally oriented split tab having halves 20a and 20b extending from a resilient cylindrical hinge 22a defining one side of the hub 22. FIGS. 13a-13d show the embodiment in an opened configuration where hinge 22a has been flexed to open an opposing slot 35 in sheath 12 beginning at the base of tabs 20a and 20b and extending along the longitudinal length of sheath 12. The embodiments of FIGS. 12a-13d are characterized by the openable slot 35 being located at the tab-to-hub connection of the split tab halves 20a and 20b.

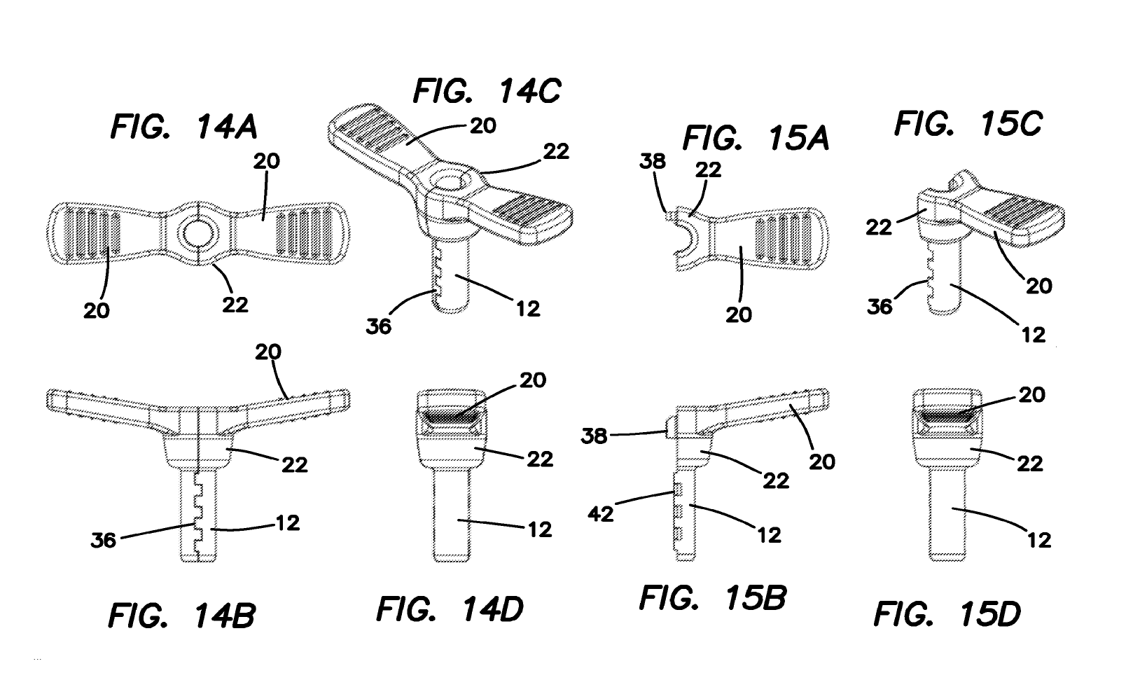

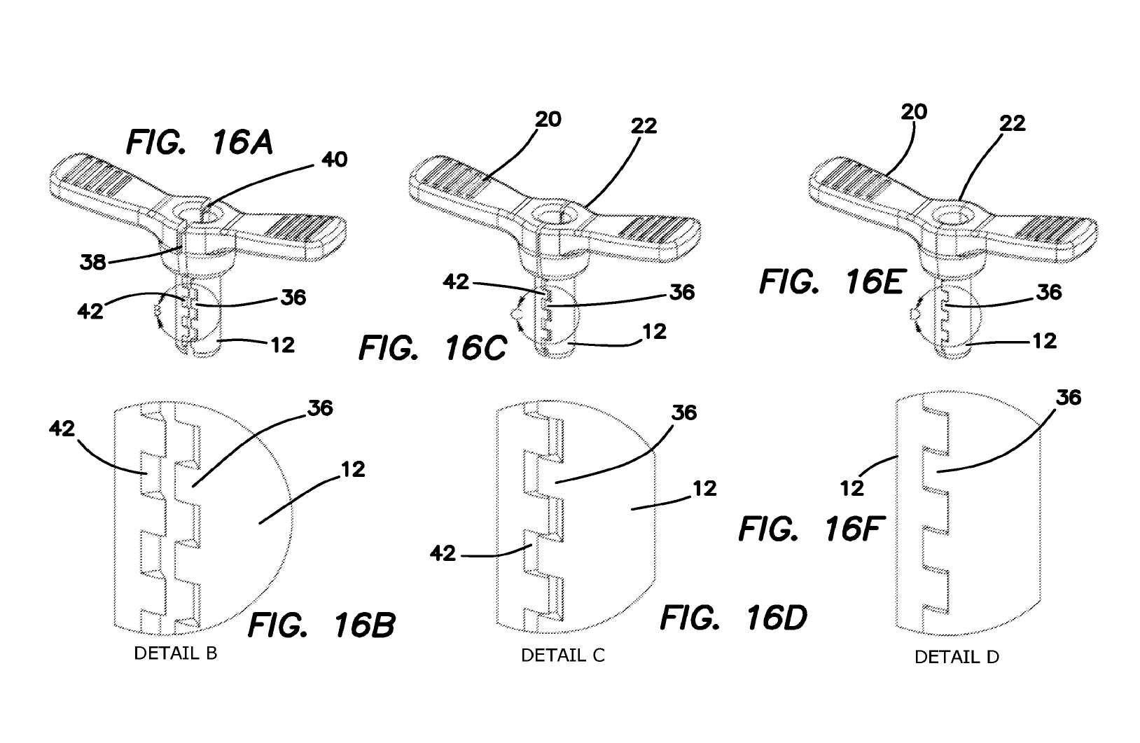

FIGS. 14a-14d show an embodiment of the invention with a plurality of interlocking or interlaced angled projections 37 defined in the hub 22 and down the longitudinal axis of the sheath 12. A pair of fingers 38 are formed on the inner surface of a hub portion 22 as best shown in FIG. 15a which provide an interference fit into a mating indentation 40 partially shown in FIG. 16a in the opposing inner surface of the opposing hub portion. The fit of the mating fingers 38 with the indentations 40 determine the force which is required to separate the TVI for a given material or combination of materials. Similarly, interlocking angled projections 36 on sheath 12 mate into conforming angled indentations 42 defined in the edge of the opposing surface of sheath 12 as shown in FIGS. 16b, 16d and 16f. These interlocking angled projections 36 and angled indentations 42 in the sheath 12 serve to keep the sheath from becoming misaligned when the two halves are assembled together. The interlocking sections also keep the sheath 12 from becoming misaligned when it is inserted into and through an introducer hemostatic valve. The number of interlocking projections can be as few as one or as many as can fit along the length of the sheath 12. The interface between the interlocking sections is angled so as to prevent the two halves of the sheath 12 from sliding and misaligning in any plane perpendicular to the assembled direction. It is important that the sheath 12 stays aligned when assembled so as to maintain an obstruction free path for delivery of a lead or vascular device through the valved introducer. A longer and more flexible sheath 12 will need the interlocking feature more than a shorter more rigid sheath 12 that is held in alignment by the hub 22. Alternate embodiments are also contemplated for the sheath 12 that prevent misalignment of the sheath in order to maintain an obstruction free path for delivery of a lead or vascular device through the valved introducer. Mechanical means such as tabs, angled surfaces, keyways, detents, fingers, frictional and temporary adhesive, magnets, static attraction, joined edges, and or any other known means of preventing the two opposing sheath surfaces from freely sliding on each other when assembled.

Any of the discussed embodiments for the sheath 12 that prevent misalignment of the sheath in order to maintain an obstruction free path for delivery of a lead or vascular device through the valved introducer can be added to any of the TVI embodiments provided in this disclosure to prevent the sheath 12 from becoming misaligned.

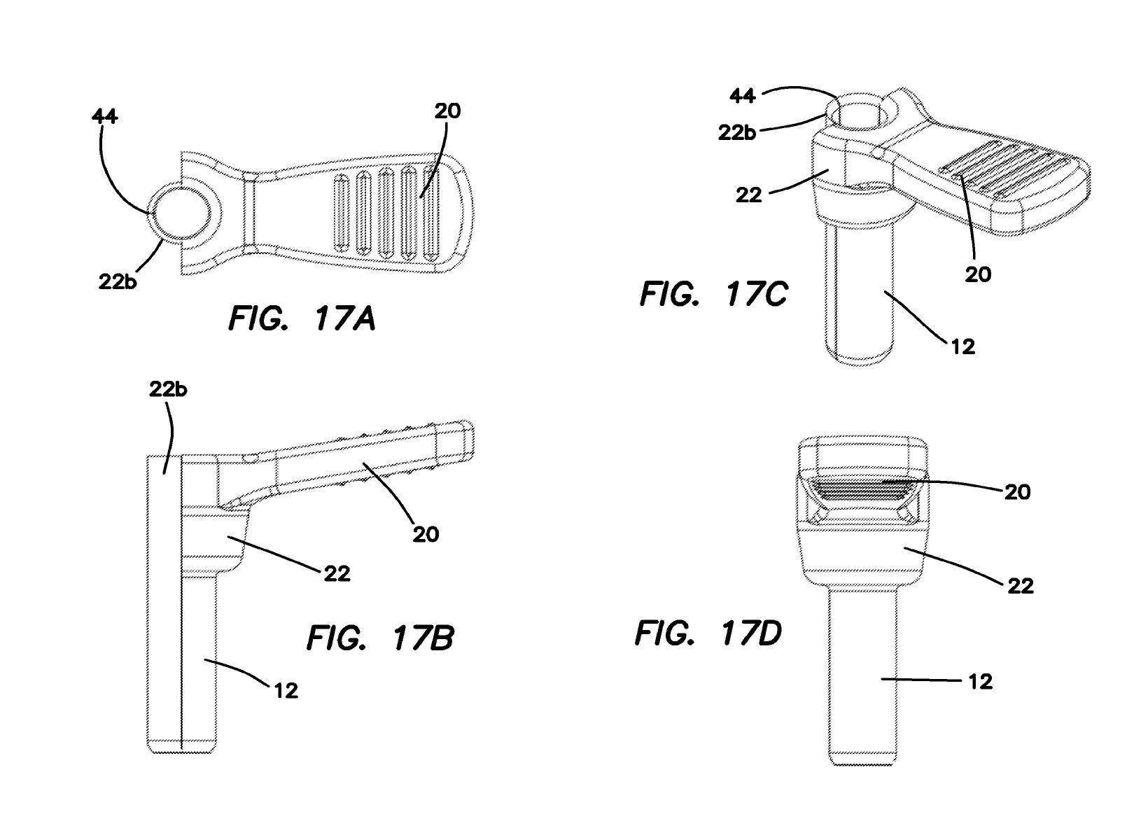

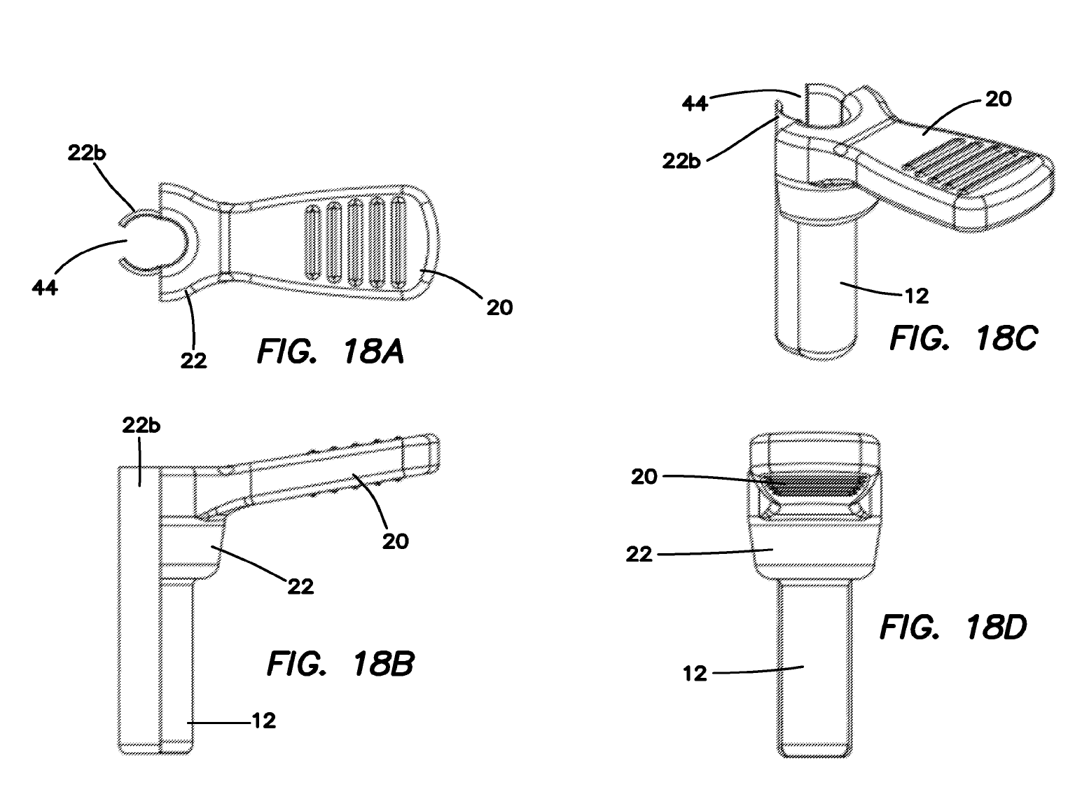

FIGS. 17a-17d are directed to an embodiment of the invention with a single horizontally oriented tab 20 extending from a separable cylinder defining one side of the hub 22b. In the preferred embodiment a slit 44 is defined longitudinally down the midline of portion 22b and extending into sheath 12. FIGS. 18a-18d show the embodiment of FIGS. 17a-17d with slit 44 opened, which may be accomplished, for example, by pulling the intravascular tool through slit 44 as TVI 10 is removed from the hemostatic valve. It is also contemplated that slit 44 be angled or stepped to prevent the hub 22b from collapsing and obstructing the free path formed by the hub 22b and the sheath 12. Slit 44 may be entirely cut through or may be partially cut through leaving a very thin membrane which is easily ruptured when the intravascular tool is pull through it.

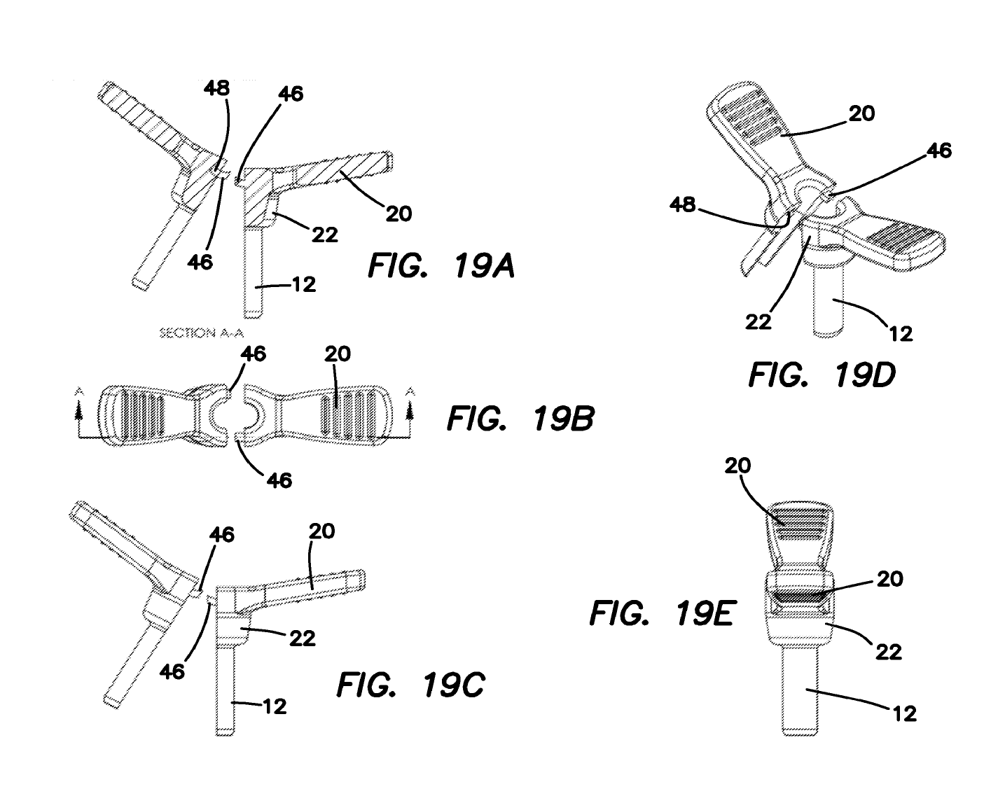

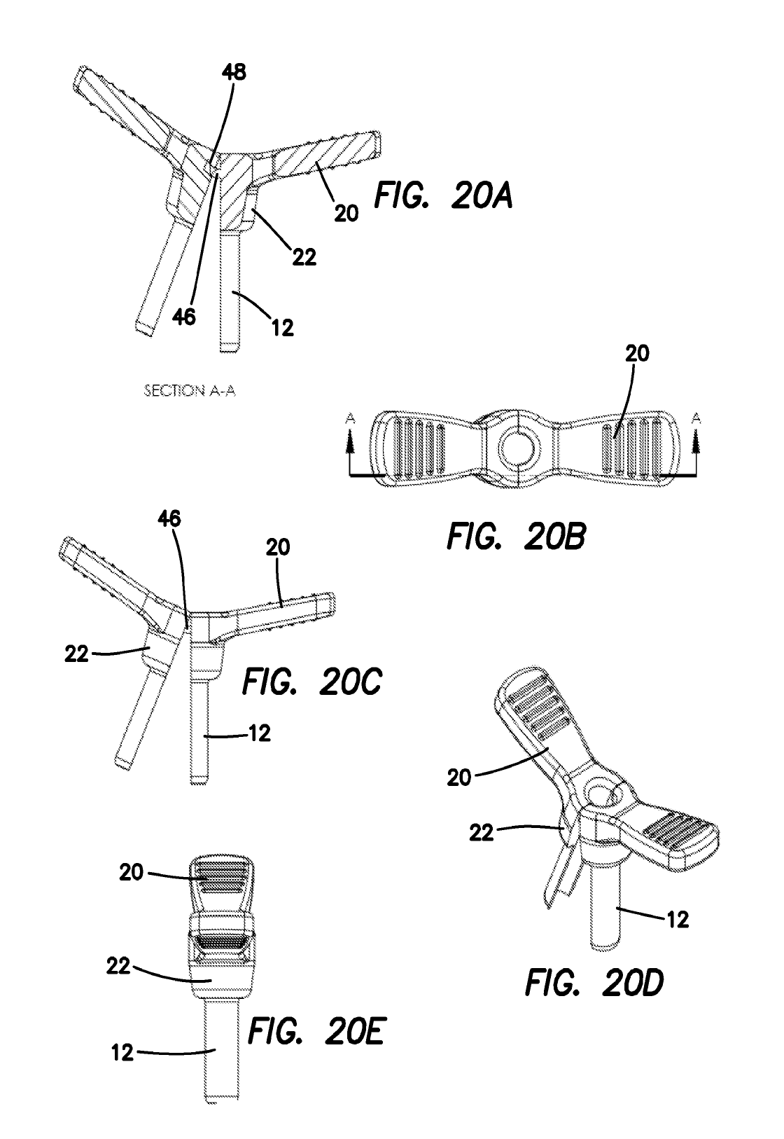

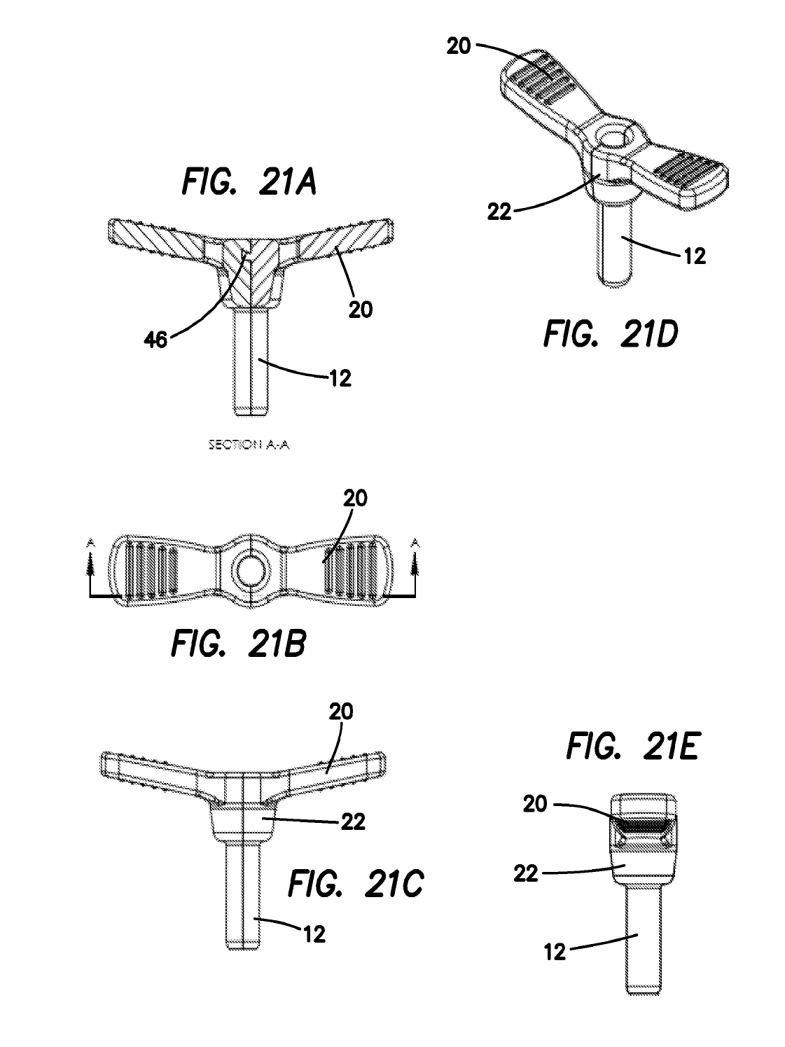

FIGS. 19a-19e are directed to an embodiment of the invention with a one way swing latch 46 defined in the hub 22 shown in a fully separated configuration. A curved latching finger 46 on each hub portion extends into a mating curved cavity 48 defined into the inner surface of the opposing hub portion like a curved tenon and mortise combination. The hub portions are engaged by rotating them together so that the curved fingers 46 are fully inserted into cavities 48 as shown in FIGS. 20a-20e in a partially assembled configuration as the swing latch is partially still open but is being closed and in FIGS. 21a-21e in the fully assembled configuration after the swing latch is fully closed. The two portions of sheath 12 will flushly and freely be disposed adjacent to each other.

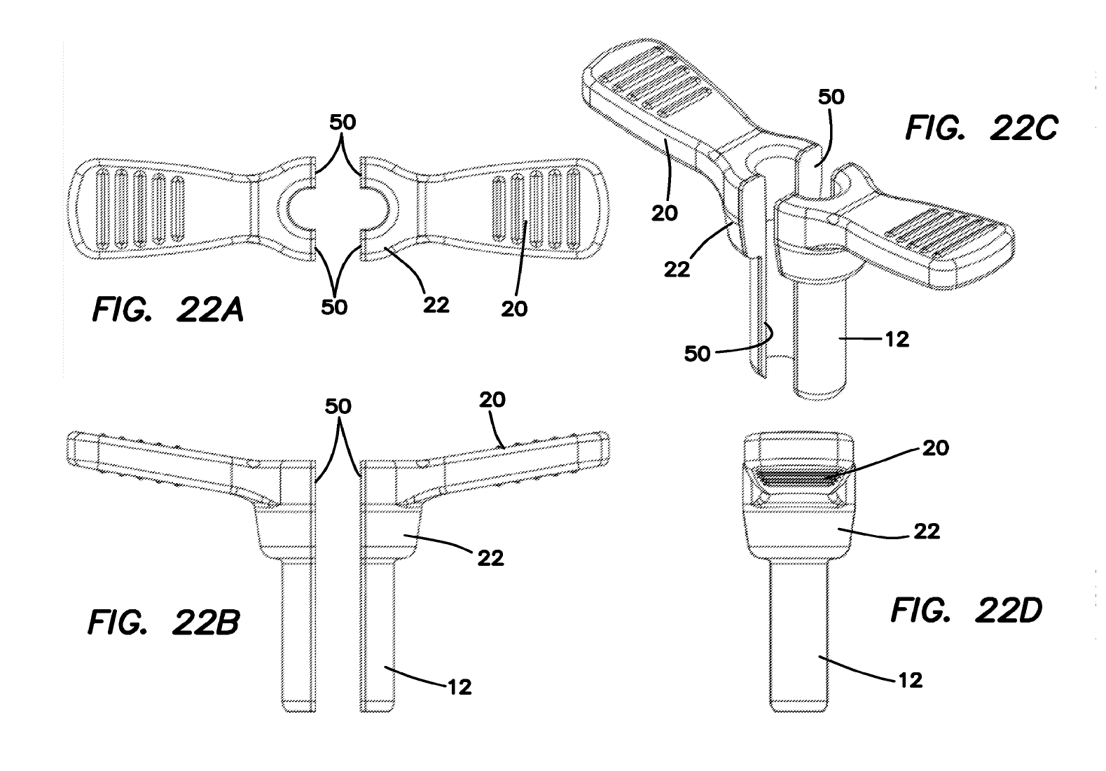

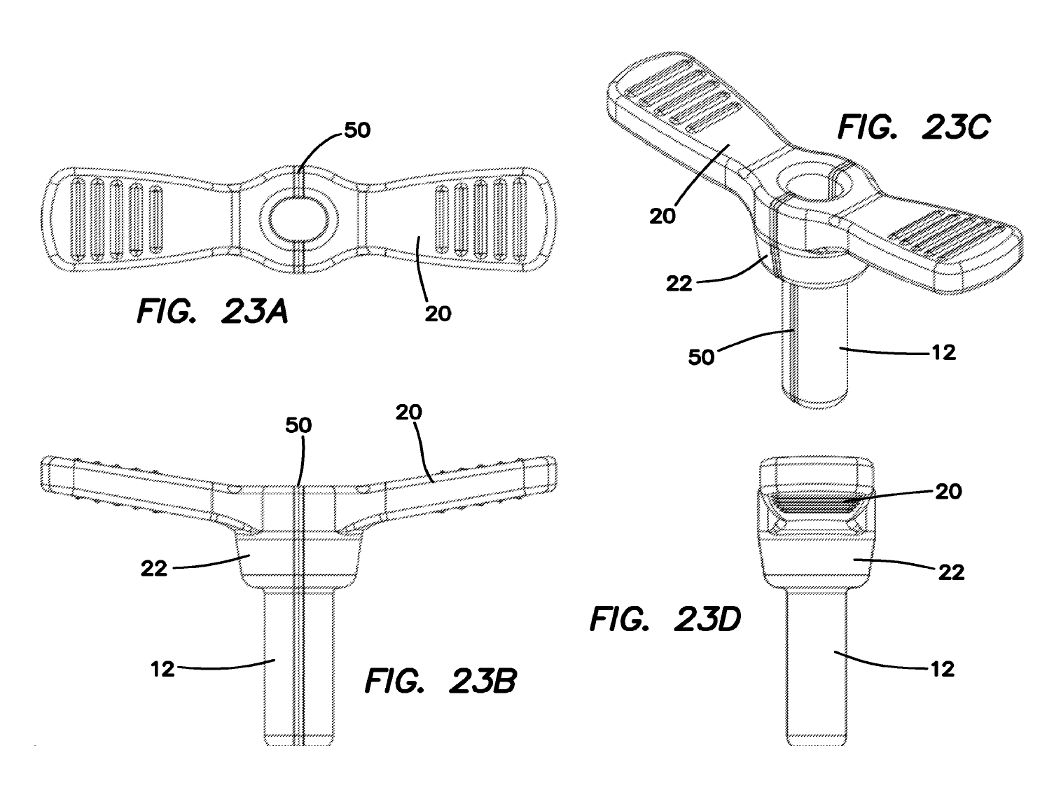

FIGS. 22a-22e are directed to an embodiment of the invention with a longitudinal resealable or adhesive membrane 50 disposed on the inner surfaces of the hub 22 and sheath 12 shown in a fully unsealed configuration. Membrane 50 can being sealed or joined together by pressure and then easily pulled apart with the capability of being rejoined an indefinite number of times. The two portions of the hub 22 and sheath 12 are flushly and freely disposed adjacent to each and temporarily join together by virtue of the adhesive quality of the membrane 50. FIGS. 23a-23e show the embodiment when joined together by bringing the membrane-bearing portions into contact with each other.

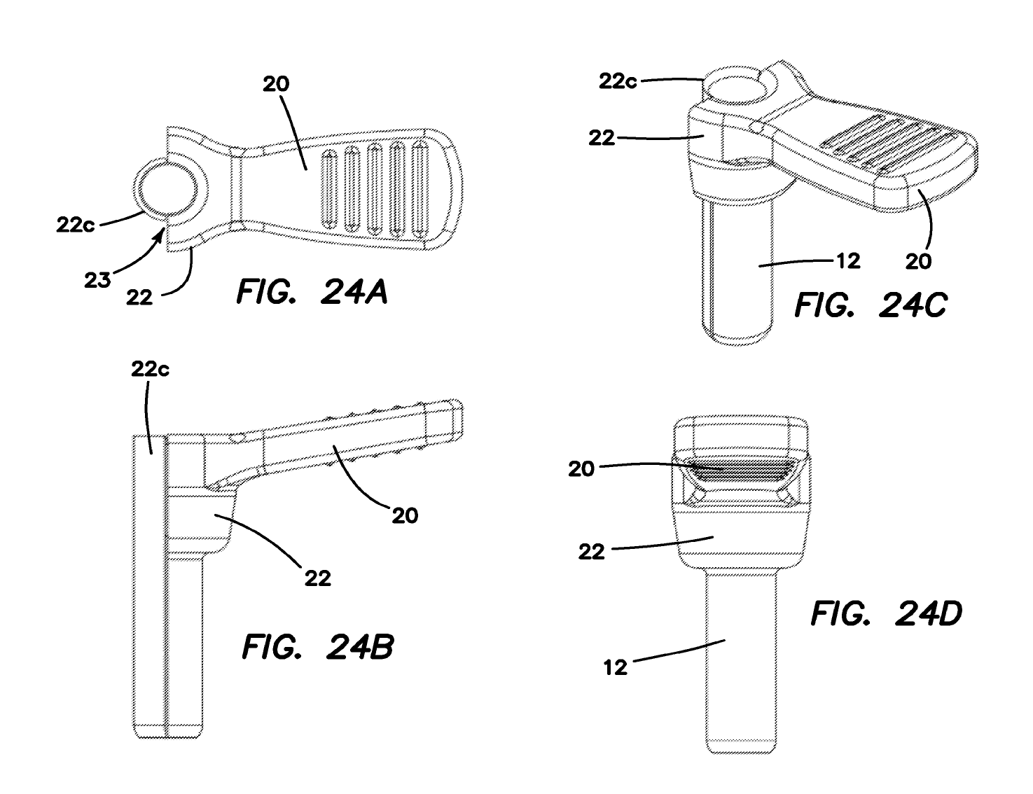

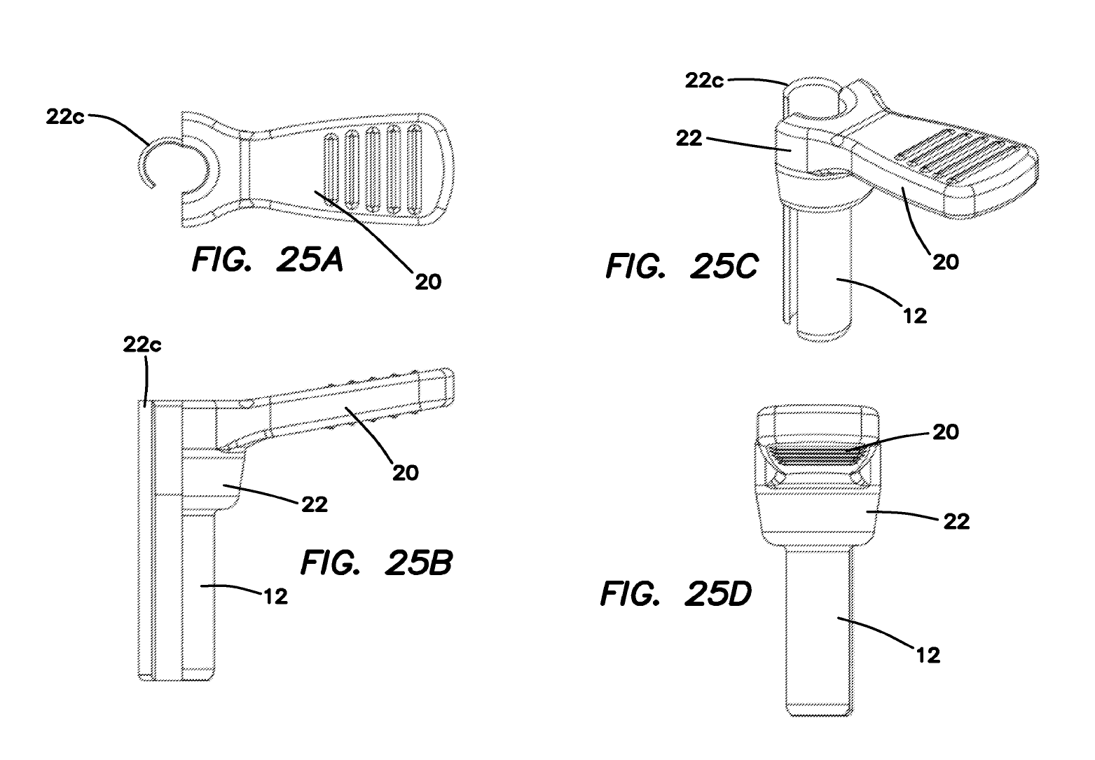

FIGS. 24a-24d show an embodiment of the invention with a single horizontally oriented tab 20 extending from a cylinder 22c defining one side of the hub 22, which cylinder 22c is separable on one side edge 23 of the cylinder 22c sheath 12 or both shown in an attached configuration. Cylinder 22c attaches on one side of the cylinder 22c to the inner surface of the opposing hub portion. A separable slit or edge 23 may be defined on the detachable side or the slit 23 may be defined through cylinder 22c and retained in the closed configuration by the resiliency of the material composing cylinder 22c. The slit 23 extends longitudinally along the entire hub length and continues to the distal end of the sheath 12. FIGS. 25a-25d show the embodiment in the opened configuration.

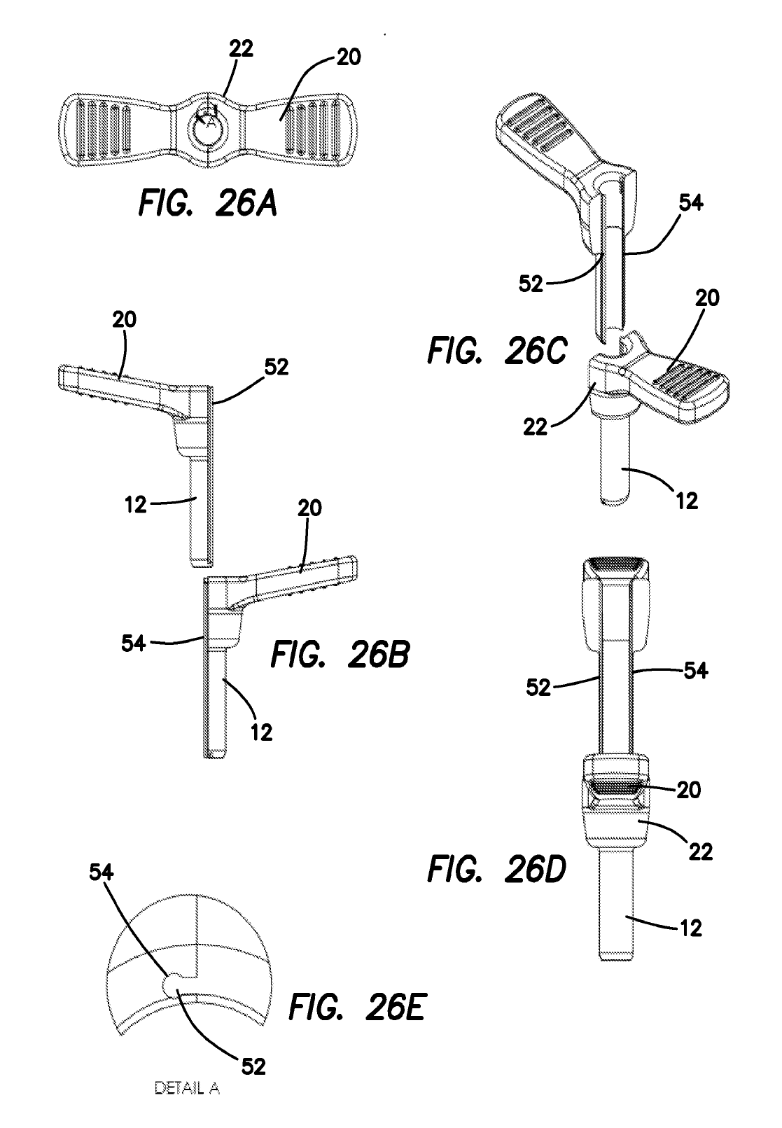

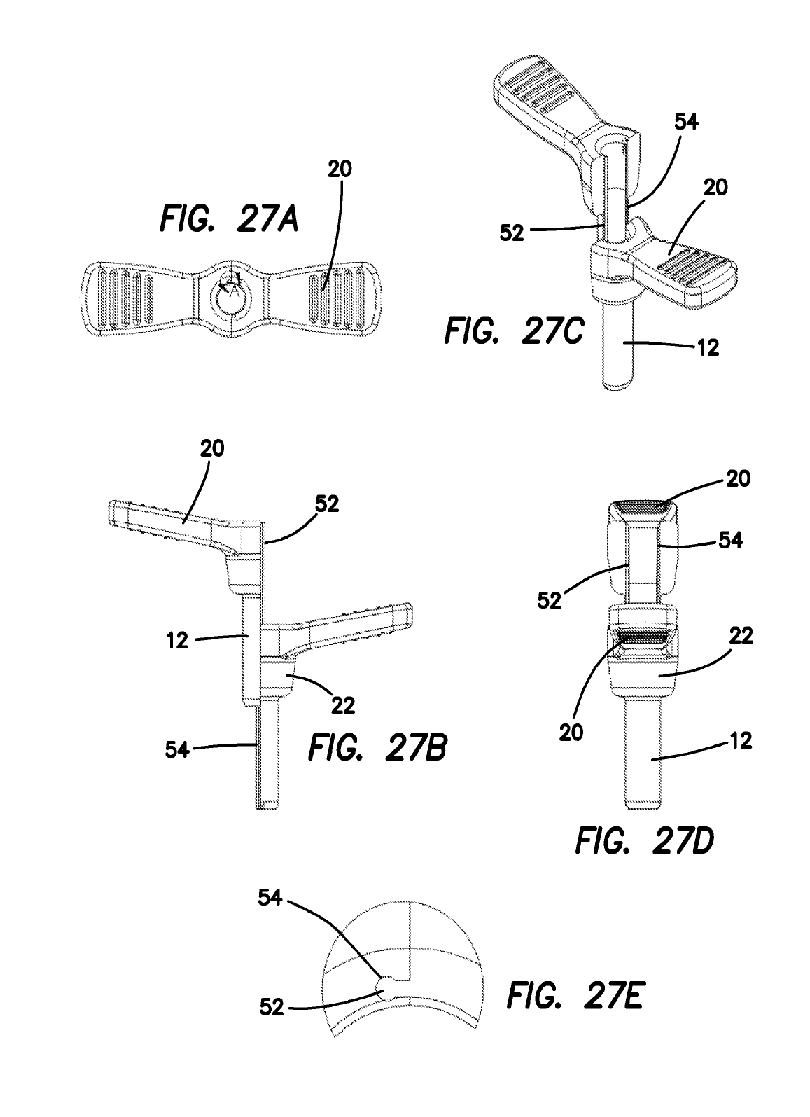

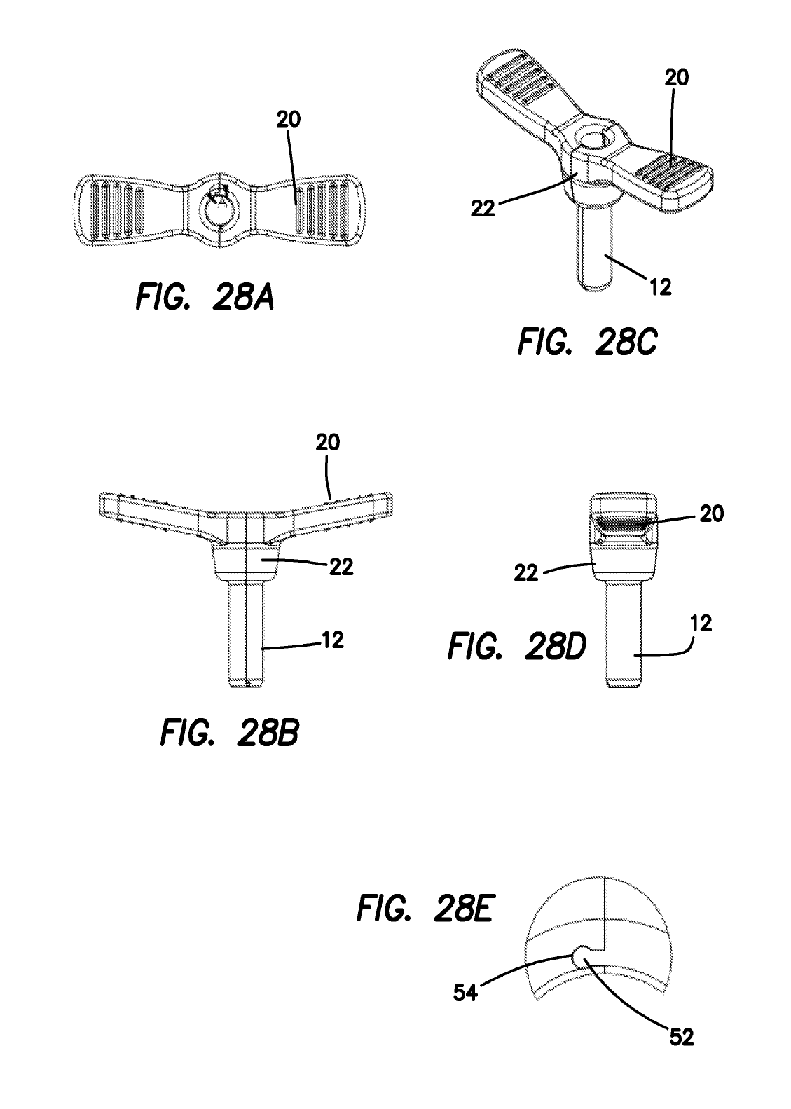

FIGS. 26a-26e are directed to an embodiment of the invention with an edge defined tongue and groove attachment which is longitudinally defined through the hub 22 and sheath 12 and in which the two opposing portions longitudinally slide or snap together and apart from each other and shown in a detached configuration. A longitudinally extending tongue 52 slides longitudinally in or snaps into a longitudinally extending groove 54 as best seen in the detailed enlarged view of FIG. 26e. Tongue 52 has a bulbous or cylindrical distal end to provide a captured configuration when slid or snapped into a mating and conforming groove 54. FIGS. 27a-27e show the embodiment partially assembled or disassembled and FIGS. 28a-28e show the embodiment fully assembled.

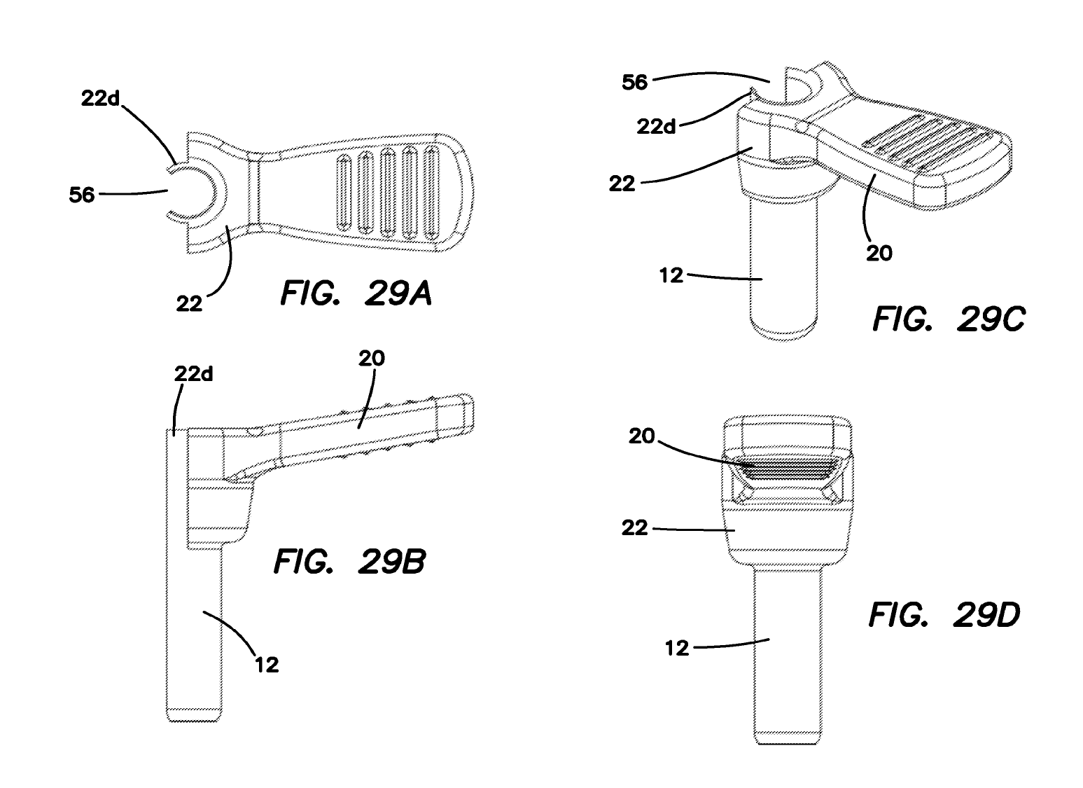

FIGS. 29a-29d are directed to an embodiment of the invention with a single horizontally oriented tab 20 wherein an open slot 56 is defined in an opposing cylinder 22d portion of the hub 22 and continues longitudinally along the longitudinal axis of the sheath 12. The slot 56 continues along the longitudinal length of hub 22 and sheath 12 to its distal end.

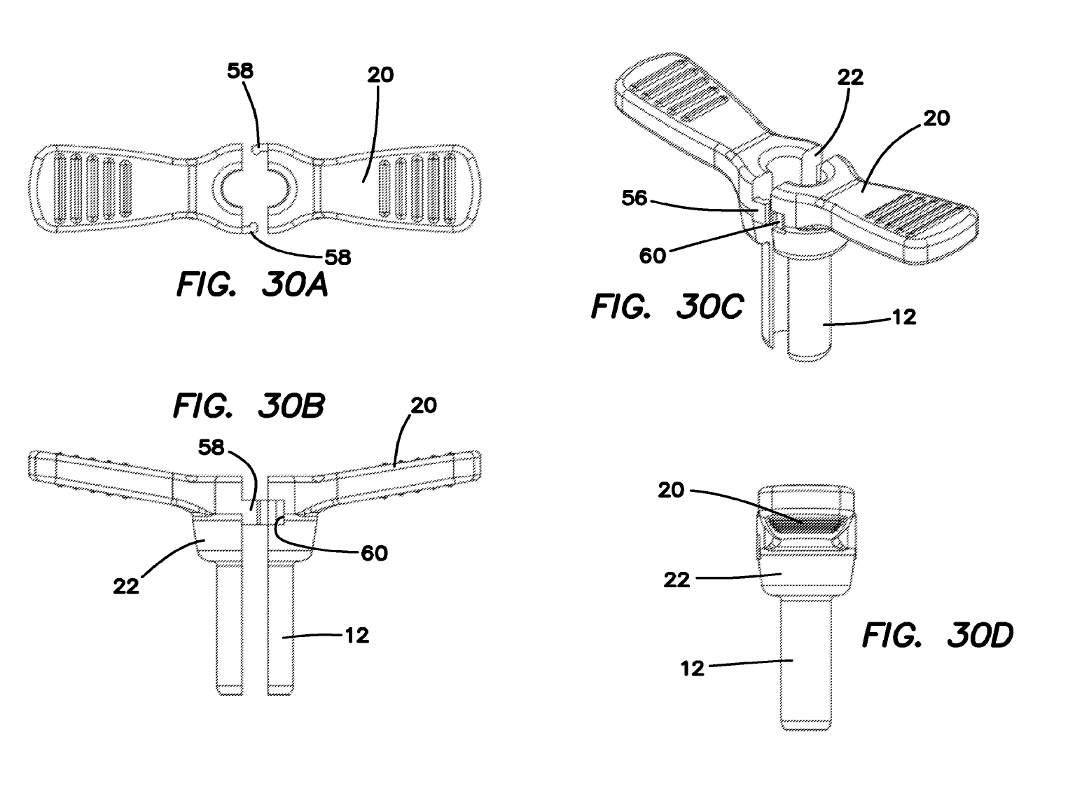

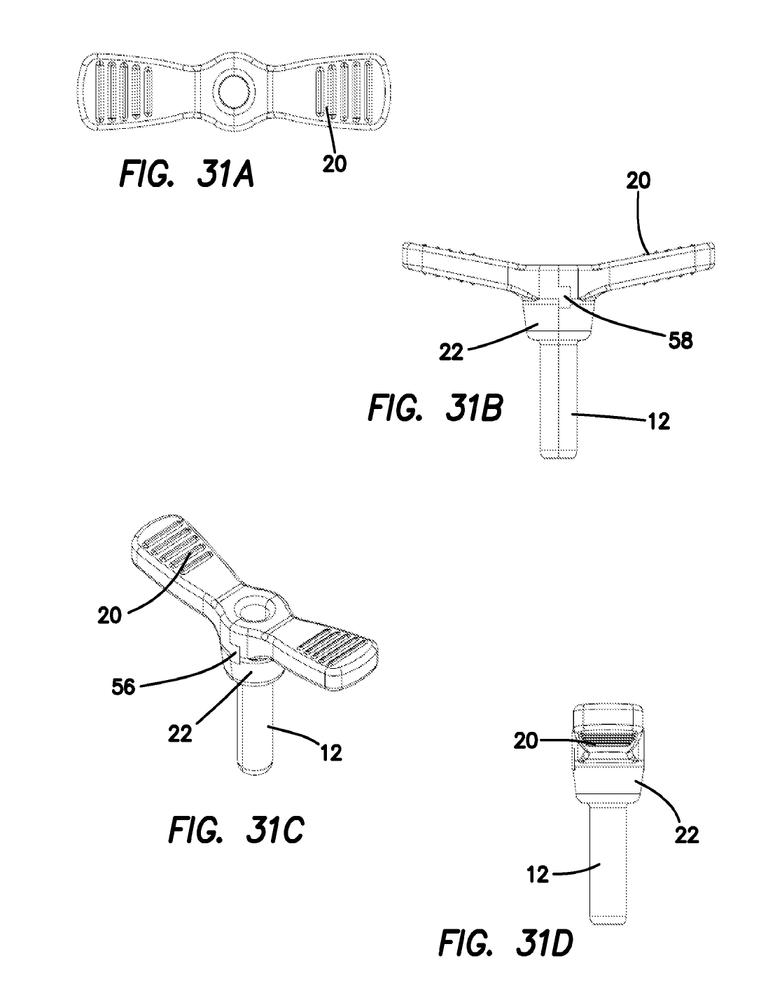

FIGS. 30a-30d show an embodiment of the invention with a pair of opposing male snap fingers 58 and mating female recesses 60 defined in the mid portion of the hub 22 and shown in an unassembled configuration. Finger 58 is illustrated in FIG. 30c as being integrally formed at opposing sides of a middle position of hub 22 disposed over a portion of the upper and lower halves of hub 22 and extending below the plane of tabs 20, however, it is to be understood that other positions on hub 22 could be utilized. The embodiment is shown in a fully assembled configuration in FIGS. 31a-31d. The sheath 12 is formed into two portions which are flushly and freely disposed together along their longitudinal length.

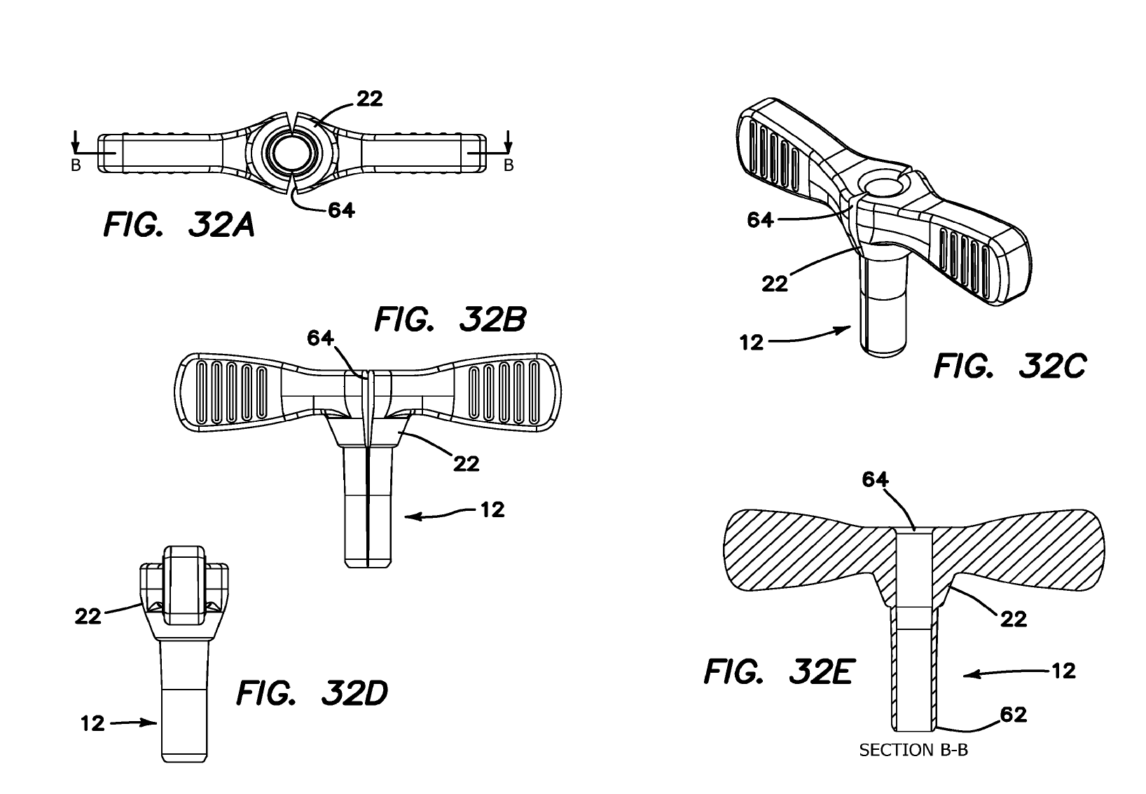

FIGS. 32a-32e are directed to an embodiment of the invention with an edge defined line of weakness, which is longitudinally defined through the integrally molded hub 22 and integrally molded sheath 12 and in which the two opposing portions are longitudinally attached and tear apart from each other. In this embodiment, sheath 12 has a completely smooth exterior surface along its entire length. Distal end 62 is intended to provide the mechanical stop against the bottom or other portion of the hemostatic valve capable of serving as a stop, thereby preventing TVI 10 from being inserted any further into the mating hemostatic valve. In the embodiment of FIGS. 32a-32e TVI 10 is an integrally molded piece, and the weakening through hub 22 and sheath 12 is provided by a pair of opposing molded V-grooves 64 which are defined in both hub 22 and sheath 12. V-groove 64 is deep enough to leave only a thin easily tearable membrane connecting the halves of the TVI 10, but not so deep as to completely penetrate the material into which it is defined. The line of weakening in sheath 12 may also be provided by any other means known to the art as discussed above.

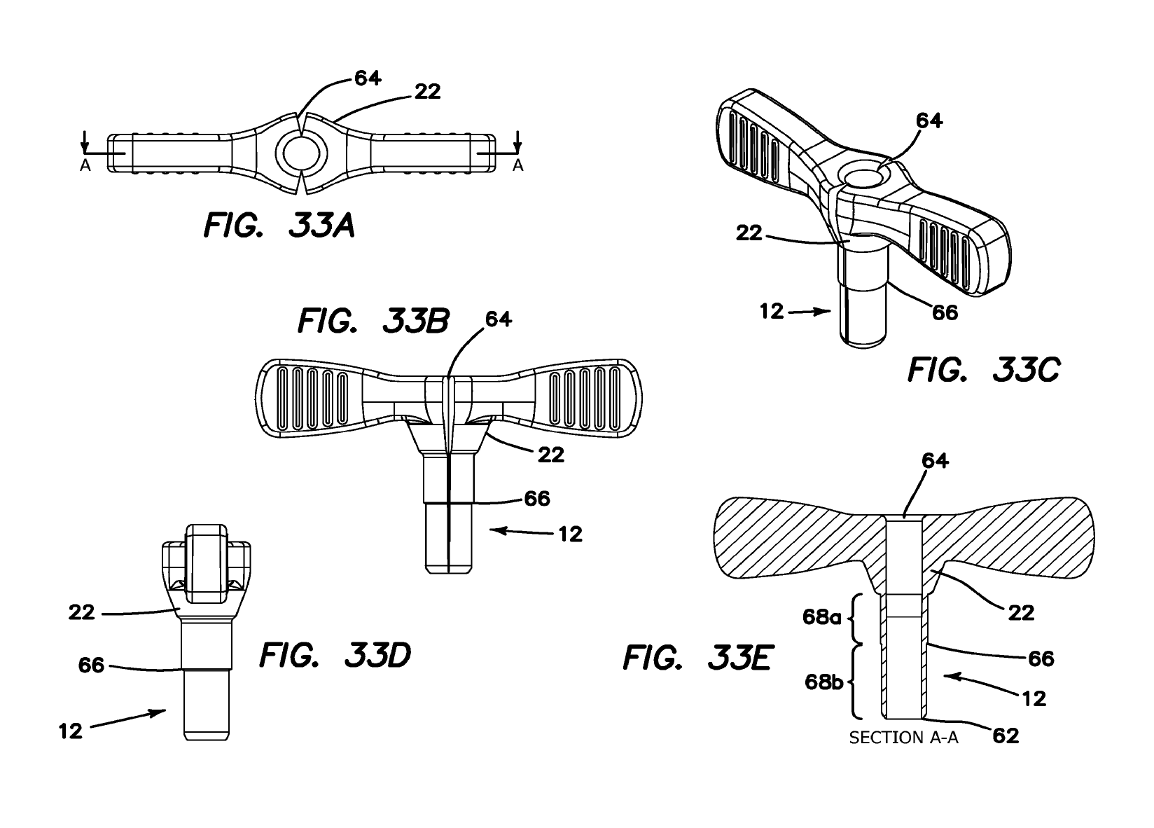

FIGS. 33a-33e are directed to an embodiment of the invention with an edge defined line of weakness which is longitudinally defined through the hub 22 and sheath 12 and in which the two opposing portions are longitudinally attached and tear apart from each other. The embodiment of FIGS. 33a-33e is similar in all respects to that embodiment shown in FIGS. 32a-32e with the exception that a molded shoulder 66 is defined in sheath 12. Shoulder 66, extending from hub 22 to a predetermined position down sheath 12, has an external diameter slightly larger than the more distal remaining portion of sheath 12. As shown in FIG. 33e sheath 12 has a proximal portion 68a of a first external diameter and a more distal remaining portion 68b having a second smaller diameter. Shoulder 66 is designed then to serve as a stop against an corresponding stop structure in the mating hemostatic valve into which TVI 10 is intended to be inserted. In such a case, distal end 62 would then not necessarily serve as the stop element.

It is to be clearly understood that TVI tool 10 need only be rigid to the extent that sheath 12 will not deform when the distal end of the sheath 12 stops against the inside of the introducer valve housing.

TVI tool 10 can be used in conjunction with any valved introducer to provide a separable unobstructed conduit through the valve. One of the unique features is the ability of TVI tool 10 to bottom out on the introducer valve housing, which allows the user to be generically instructed or directed to push the universal TVI tool through the introducer valve until it stops.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A transvalvular insertion tool for providing a passage through a hemostatically valved introducer comprising:
at least one tab;
a hub coupled to the tab;
a longitudinally rigid sheath for insertion through the valved introducer to provide an obstruction free path for delivery of a lead or vascular device through the valved introducer; and
means for separating the hub and rigid sheath to allow removal from the lead or device without removal of the transvalvular insertion tool over either end of the lead or vascular device.

2. The transvalvular insertion tool of claim 1 where the means for separating comprises longitudinal halves of the rigid sheath and a temporary mechanical coupling for selectively retaining the longitudinal halves together.

3. The transvalvular insertion tool of claim 1 where the means for separating comprises a longitudinal line of separation of the rigid sheath and a clam-shell mechanism for spreading the longitudinal line of separation of the rigid sheath apart to selectively create a longitudinal gap in the rigid sheath.

4. The transvalvular insertion tool of claim 1 where the means for separating comprises at least one longitudinal line of weakness in the rigid sheath.

5. The transvalvular insertion tool of claim 4 where the rigid sheath includes an external stop positioned between the hub and distal end of the sheath.

6. A universal transvalvular insertion tool for providing a passage through a multiplicity of differently designed hemostatically valved introducers for a lead or vascular device, each valved introducer having a valve within a valved body, the universal transvalvular insertion tool comprising:
a longitudinally rigid sheath having a lumen for insertion through the valve of any one of the multiplicity of differently designed hemostatically valved introducers to provide a substantially obstruction free path for delivery of a lead or vascular device through the valve and valved body of each of the valved introducers, the rigid sheath having a length selected to extend through the valve without substantial deformation of the lumen of the rigid sheath; and
means for separating the rigid sheath to allow removal from the lead or device without removal of the transvalvular insertion tool over either end of the lead or vascular device.

7. The tool of claim 6 further comprising a hemostatic introducer to provide a kit.

8. The tool of claim 6 where the sheath and/or hub separate into two equal halves.

9. The tool of claim 6 where the sheath and/or hub includes a living hinge, that separates into two connected equal halves.

10. The tool of claim 6 where the sheath and/or hub is arranged and configured to be separated and put back together with or without an intravascular tool being inserted therethrough at the time of recombination for indefinite repeated usage on each instance of application if desired.

11. The tool of claim 6 where the sheath and/or hub has a plurality of interlocking angled projections along the sheath and/or hub to prevent the sheath and/or hub halves from becoming misaligned from forces exerted thereon by the valved introducers.

12. The tool of claim 6 where the sheath and/or hub includes a sealing member or gasket between the halves of sheath and/or hub that seals the halves to each other when assembled, which sealing member or gasket is softer than the halves of the sheath and/or hub.

13. The tool claim 6 where the sheath need only be rigid to the extent that the sheath will not deform when the distal end of the sheath stops against the inside of the introducer valve housing.

14. The tool of claim 6 where the tool is arranged and configured to be used in conjunction with any valved introducer to provide a separable unobstructed conduit through the valve.

15. The tool of claim 6 where the rigid sheath is arranged and configured to bottom out on the valved body, which allows a user to be instructed or directed to push the tool through the valve until it stops.

16. The tool of claim 1 where the means for separating the rigid sheath and/or hub to allow removal from the lead or device without removal of the transvalvular insertion tool over either end of the lead or vascular device further comprises means for reassembling of the transvalvular insertion tool.

17. The tool of claim 16 where the sheath and/or hub has two opposing portions and where the means for separating and allowing reassembly comprises fingers with an interference fit into a mating indentation in the opposing sheath and/or hub portions.

18. The tool of claim 16 where the sheath and/or hub has two opposing portions and where the means for separating and allowing reassembly comprises a snap peg and hole combination for coupling the two sheath and/or hub portions together.

19. The tool of claim 16 where the means for separating and allowing reassembly comprises a hinged clam shell mechanism having tabs and an opening on an exterior surface of the hub opposing the tabs of the clam shell mechanism.

20. The tool of claim 16 where the means for separating and allowing reassembly comprises an insert disposed in the hub and a tongue and groove combination defined at least in part in the insert disposed in the hub.

21. The tool of claim 16 where the means for separating and allowing reassembly comprises a single split tab with an opposing resilient hub portion having a longitudinal slit and sheath defined inwardly at least in the hub between the split portions of the tab to provide a clam shell mechanism for opening the hub.

22. The tool of claim 16 where the means for separating and allowing reassembly comprises a plurality of snap interlaced fingers and recesses longitudinally defined in opposing portions of the sheath and a tenon and mortise combination defined in an inner surface of the opposing hub portions.

23. The tool of claim 16 where the means for separating and allowing reassembly comprises a single tab and an opposing split cylinder portion forming the hub and sheath.

24. The tool of claim 16 where the means for separating and allowing reassembly comprises a one-way swing latch combination defined in an inner portion of the hub portions.

25. The tool of claim 16 where the means for separating and allowing reassembly comprises a resealable membrane combination disposed longitudinally along the hub and/or sheath.

26. The tool of claim 16 where the means for separating and allowing reassembly comprises a single tab having a half cylinder defined therein and an opposing half cylinder to form the hub and sheath and with a hinge on one side of the opposing half cylinder coupled to the tab and with a split defined opposing the hinge and extending longitudinally along the sheath.

27. The tool of claim 16 where the means for separating and allowing reassembly comprises a slidable, longitudinal tongue and groove combination defined in the hub and/or sheath.

28. The tool of claim 16 where the means for separating and allowing reassembly comprises a single tab and an opposing cylinder forming the hub and sheath with an open slot longitudinally defined therein and extending along the hub and sheath.

29. The tool of claim 16 where the means for separating and allowing reassembly comprises opposing portions of the hub having opposing sides and a snap finger and recess combination defined in the opposing sides of the sheath and/or hub.

30. A method of using a transvalvular insertion tool for providing a passage through a hemostatically valved introducer comprising:
inserting a longitudinally rigid sheath through the valved introducer to provide an obstruction free path for delivery of a lead or vascular device through the valved introducer; and
separating the hub and rigid sheath to allow removal from the lead or device without removal of the transvalvular insertion tool over either end of the lead or vascular device.

31. The method of claim 30 further where inserting a longitudinally rigid sheath through the valved introducer is performed until the tool is stopped in the valved introducer.

32. The method of claim 30 further comprising reassembling the hub and sheath after use for a repeated use.
